(19) United States
(12) Patent Application Publication
(10) Pub. No.: US 2009/0215805 A1
(43) Pub. Date: Aug. 27, 2009

(54) 4-Heteroaryl Pyrimidine Derivatives and use thereof as Protein Kinase Inhibitors

(75) Inventors: Gavin Wood, Cupar Fife (GB); Christopher Meades, Dundee (GB); Peter Martin Fischer, Nottingham (GB); Shudong Wang, Nottingham (GB); Kenneth Duncan, Waltham, MA (US); Daniella I. Zheleva, Fife (GB); Campbell Mcinnes, Irmo, SC (US); Mark Thomas, Dundee (GB)

Correspondence Address: LAHIVE & COCKFIELD, LLP;FLOOR 30, SUITE 3000, ONE POST OFFICE SQUARE, BOSTON, MA, 02109, US

(73) Assignee: Cyclacel Limited, Dundee (GB)

(21) Appl. No.: 12/083,559

(22) PCT Filed: Oct. 9, 2006
(86) PCT No.: PCT/GB2006/003751

§ 371 Date: Feb. 12, 2009

(30) Foreign Application Priority Data
Oct. 14, 2005 (GB) 0520955.6

(51) Int. Cl. A61K 31/506; C07D 239/02; G01N 33/566; G01N 33/53; A61P 25/28; A61P 3/10; A61P 35/00

(52) U.S. Cl. 514/275; 544/297; 436/501; 435/7.1

(57) ABSTRACT

The present invention relates to compounds of formula (I) or formula (II), or pharmaceutically acceptable salts thereof. Further aspects relate to pharmaceutical compositions comprising compounds according to the invention, and the use of said compounds in the preparation of a medicament for treating a variety of disorders, including proliferative disorders, viral disorders, stroke, etc.

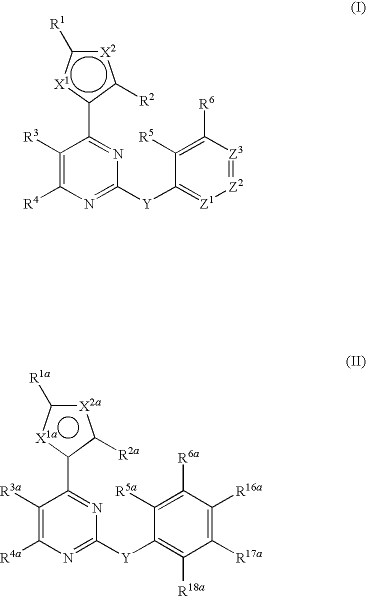

The present invention relates to substituted pyrimidine derivatives. In particular, the invention relates to 2-substituted-4-heteroaryl-pyrimidines and their use in therapy. More specifically, but not exclusively, the invention relates to compounds that are capable of inhibiting one or more protein kinases.

### BACKGROUND TO THE INVENTION

In eukaryotes, all biological functions, including DNA replication, cell cycle progression, energy metabolism, and cell growth and differentiation, are regulated through the reversible phosphorylation of proteins. The phosphorylation state of a protein determines not only its function, subcellular distribution, and stability, but also what other proteins or cellular components it associates with. The balance of specific phosphorylation in the proteome as a whole, as well as of individual members in a biochemical pathway, is thus used by organisms as a strategy to maintain homeostasis in response to an ever-changing environment [Cohen, P. Nat. Rev. Drug Disc., 2002, 1, 309]. The enzymes that carry out these phosphorylation and dephosphorylation steps are protein kinases and phosphatases, respectively. Many kinases have gained importance as drug discovery targets in a variety of therapeutic areas [Fischer, P. M. Curr. Med. Chem., 2004, 11, 1563].

The eukaryotic protein kinase family is one of the largest in the human genome, comprising some 500 genes [Manning, G;. Whyte, D. B.; Martinez, R.; Hunter, T.; Sudarsanam, S., The protein kinase complement of the human genome, Science 2002, 298, 1912-1934; Kostich, M.; English, J.; Madison, V.; Gheyas, F.; Wang, L., et al. Human members of the eukaryotic protein kinase family, Genome Biology 2002, 3, Research 0043.0041-0043.0012].

The majority of kinases contain a 250-300 amino acid residue catalytic domain with a conserved core structure. This domain comprises a binding pocket for ATP (less frequently GTP), whose terminal phosphate group the kinase transfers covalently to its macromolecular substrates. The phosphate donor is always bound as a complex with a divalent ion (usually Mg²⁺ or Mn²⁺). Another important function of the catalytic domain is the binding and orientation for phosphotransfer of the macromolecular substrate. The catalytic domains present in most kinases are more or less homologous.

A wide variety of molecules capable of inhibiting protein kinase function through antagonising ATP binding are known in the art [Dancey, J.; Sausville, E. A. Issues and progress with protein kinase inhibitors for cancer treatment, Nat. Rev. Drug Disc. 2003, 2, 296-313; Cockerill, G. S.; Lackey, K. E., Small molecule inhibitors of the class 1 receptor tyrosine kinase family. Current Topics in Medicinal Chemistry 2002, 2, 1001-1010; Fabbro, D.; Ruetz, S.; Buchdunger, E.; Cowan-Jacob, S. W.; Fendrich, G. et al., Protein kinases as targets for anticancer agents: from inhibitors to useful drugs, Pharmacol. Ther. 2002, 93, 79-98; Cohen, P., Protein kinases—the major drug targets of the twenty-first century? Nat. Rev. Drug Disc. 2002, 1, 309-315; Bridges, A. J., Chemical inhibitors of protein kinases, Chem. Rev. 2001, 101(8), 2541-2571].

By way of example, the applicant has previously disclosed 2-anilino-4-heteroaryl-pyrimidine compounds with kinase inhibitory properties, particularly against cyclin-dependent kinases (CDKs) [Wang, S.; Meades, C.; Wood, G.; Osnowski, A.; Fischer, P. M., N-(4-(4-methylthiazol-5-yl)pyrimidin-2-yl)-N-phenylamines as antiproliferative compounds, PCT Intl. Patent Appl. Publ. WO 2003029248, Cyclacel Limited, UK; Wu, S. Y.; McNae, I.; Kontopidis, G.; McClue, S. J.; McInnes, C. et al., Discovery of a Novel Family of CDK Inhibitors with the Program LIDAEUS: Structural Basis for Ligand-Induced Disordering of the Activation Loop, Structure 2003, 11, 399-410; Fischer, P. M.; Wang, S.; Wood, G., Inhibitors of cyclin dependent kinases as anti-cancer agents, PCT Intl. Patent Appl. Publ. WO 02/079193; Cyclacel Limited, UK; Wang, S.; Fischer, P. M. Anti-cancer compounds, US Patent Appl. Publ. 2002/0019404; Fischer, P. M.; Wang, S., 2-substituted 4-heteroaryl-pyrimidines and their use in the treatment of proliferative disorders, PCT Intl. Patent Appl. Publ. WO 2001072745; Cyclacel Limited, UK].

CDKs are serine/threonine protein kinases that associate with various cyclin subunits. These complexes are important for the regulation of eukaryotic cell cycle progression, but also for the regulation of transcription [Knockaert, M.; Greengard, P.; Meijer, L., Pharmacological inhibitors of cyclin-dependent kinases, Trends Pharmacol. Sci. 2002, 23, 417-425; Fischer, P. M.; Endicott, J.; Meijer, L., Cyclin-dependent kinase inhibitors, Progress in Cell Cycle Research; Editions de la Station Biologique de Roscoff: Roscoff, France, 2003; pp 235-248].

The present invention seeks to provide further substituted pyrimidine derivatives. More specifically, the invention relates to compounds that have broad therapeutic applications in the treatment of a number of different diseases and/or that are capable of inhibiting one or more protein kinases.

### STATEMENT OF INVENTION

A first aspect of the invention relates to compounds of formula I, or pharmaceutically acceptable salts thereof,

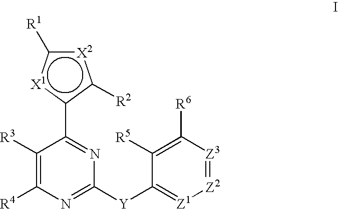

wherein
one of X¹ and X² is NR⁷ and the other is CR⁸;
one of Z¹, Z² and Z³ is N or NR⁹⁺ and the remainder are each independently CR¹⁰;
Y is selected from NR¹¹, NHCO, NHSO₂, NHCH₂, CH₂, CH₂CH₂, or CH═CH;
R¹-R⁶, R⁸ and each R¹⁰ are each independently selected from H or (CH₂)ₘR¹², where m is 0, 1, 2, or 3;
R⁷ and R¹¹ are each independently H or alkyl;
R⁹ is alkyl;
each R¹² is independently selected from OR¹³, R¹³, COR¹³, COOR³, CN, CONR¹³R¹⁴, NR¹³R¹⁴, NR¹³COR¹⁴, SR¹³, SOR¹³, SO₂R¹³, NR¹³SO₂R¹⁴, SO₂OR¹³, SO₂NR¹³R¹⁴, halogen, CF₃, and NO₂;
R¹³ and R¹⁴ are each independently H or (CH₂)ₙR¹⁵, where n is 0, 1, 2, or 3; and
each R¹⁵ is independently selected from alkyl, cycloalkyl, heteroaryl, aralkyl, aryl and heterocycloalkyl, each of which may be optionally substituted by one or more substituents selected from halogen, OH, CN, COO-alkyl, aralkyl, SO₂-alkyl, SO₂-aryl, COOH, CO-alkyl, CO-aryl, NH₂, NH-alkyl, N(alkyl)₂, CF₃, alkyl and alkoxy, wherein said alkyl and alkoxy groups may be further substituted by one or more OH groups.

A second aspect of the invention relates to compounds of formula II, or pharmaceutically acceptable salts thereof,

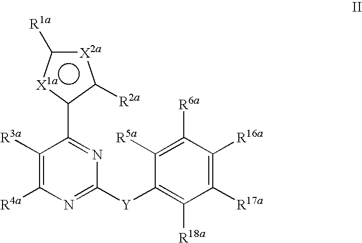

wherein
one of X^{1a} and X^{2a} is NR^{7a} and the other is CR^{8a};
Y is selected from NR^{11a}, NHCO, NHSO₂, NHCH₂, CH₂, CH₂CH₂, or CH═CH;
R^{1a}-R^{6a}, R^{8a}, R^{16a}, R^{17a} and R^{18a} are each independently selected from H or (CH₂)ₘR^{12a}, where m is 0, 1, 2, or 3;
R^{7a} and R^{11a} are each independently H or alkyl;
each R^{12a} is independently selected from OR^{13a}, R^{13a}, COR^{13a}, COOR^{13a}, CN, CONR^{13a}R^{14a}, NR^{13a}R^{14a}, NR^{13a}COR^{14a}, SR^{13a}, SOR^{13a}, SO₂R^{13a}, NR^{13a}SO₂R^{14a}, SO₂OR^{13a}, SO₂NR^{13a}R^{14a}, halogen, CF₃, and NO₂;
R^{13a} and R^{14a} are each independently H or (CH₂)ₙR^{15a}, where n is 0, 1, 2, or 3; and
each R^{15a} is independently selected from alkyl, cycloalkyl, heteroaryl, aralkyl, aryl and heterocycloalkyl, each of which may be optionally substituted by one or more substituents selected from halogen, OH, CN, COO-alkyl, aralkyl, SO₂-alkyl, SO₂-aryl, COOH, CO-alkyl, CO-aryl, NH₂, NH-alkyl, N(alkyl)₂, CF₃, alkyl and alkoxy, wherein said alkyl and alkoxy groups may be further substituted by one or more OH groups;
wherein at least one of R^{1a}-R^{6a}, R^{8a}, R^{16a}, R^{17a} and R^{18a} is selected from SO₂NR^{13a}R^{14a} and optionally substituted heterocycloalkyl.

The present invention provides compounds that are capable of inhibiting various protein kinases, including aurora kinase [Carmena, M.; Eamshaw, W. C., Nat. Rev. Mol. Cell. Biol., 2003, 4, 842], FMS-like tyrosine kinase 3 (FLT3) [Stirewalt, D. L.; Radich, J. P., Nat. Rev. Cancer, 2003, 3, 650], cyclin-dependent kinases (CDKs) [Fischer, P. M.; Endicott, J.; Meijer, L., Progr. Cell Cycle Res., 2003, 5, 235], and glycogen synthase kinase 3 (GSK3) [Cohen, P.; Goedert, M., Nat. Rev. Drug Disc., 2004, 3, 479].

Another aspect of the invention relates to pharmaceutical compositions comprising a compound according to the invention as defined above admixed with a pharmaceutically acceptable diluent, excipient or carrier.

Further aspects of the invention relate to the use of compounds of the invention as defined above in the preparation of a medicament for treating one or more of the following:
a proliferative disorder;
a viral disorder;
a CNS disorder;
a stroke;
a microbial infection;
a fungal disorder;
a parasitic disorder;
an inflammatory disorder;
a cardiovascular disorder.
alopecia; and
diabetes.

Another aspect of the invention relates to the use of compounds of the invention as defined above in an assay for identifying further candidate compounds capable of inhibiting one or more of a cyclin dependent kinase, GSK, aurora kinase, a tyrosine kinase, FMS-like tyrosine kinase-2 (FLT-3) and a PLK enzyme.

Another aspect of the invention relates to compounds of the invention as defined above, or pharmaceutically acceptable salts thereof for use in medicine.

### DETAILED DESCRIPTION

As used herein, the term “hydrocarbyl” refers to a group comprising at least C and H. If the hydrocarbyl group comprises more than one C then those carbons need not necessarily be linked to each other. For example, at least two of the carbons may be linked via a suitable element or group. Thus, the hydrocarbyl group may contain heteroatoms. Suitable heteroatoms will be apparent to those skilled in the art and include, for instance, sulphur, nitrogen, oxygen, phosphorus and silicon. Where the hydrocarbyl group contains one or more heteroatoms, the group may be linked via a carbon atom or via a heteroatom to another group, i.e. the linker atom may be a carbon or a heteroatom. Preferably, the hydrocarbyl group is an aryl, heteroaryl, alkyl, cycloalkyl, aralkyl, heterocycloalkyl, or alkenyl group. More preferably, the hydrocarbyl group is an aryl, heteroaryl, alkyl, cycloalkyl, aralkyl or alkenyl group. The hydrocarbyl group may be optionally substituted by one or more R¹² or R^{12a} groups.

As used herein, the term “alkyl” includes both saturated straight chain and branched alkyl groups which may be substituted (mono- or poly-) or unsubstituted. Preferably, the alkyl group is a C₁₋₂₀ alkyl group, more preferably a C₁₋₁₅, more preferably still a C₁₋₁₂ alkyl group, more preferably still, a C₁₋₆ alkyl group, more preferably a C₁₋₃ alkyl group. Particularly preferred alkyl groups include, for example, methyl, ethyl, propyl, isopropyl, butyl, isobutyl, tert-butyl, pentyl and hexyl. Suitable substituents include, for example, one or more R¹² or R^{12a} groups. Preferably, the alkyl group is unsubstituted.

As used herein, the term “cycloalkyl” refers to a cyclic alkyl group which may be substituted (mono- or poly-) or unsubstituted. Preferably, the cycloalkyl group is a C₃₋₁₂ cycloalkyl group. Suitable substituents include, for example, one or more R¹² or R^{12a} groups.

As used herein, the term “alkenyl” refers to a group containing one or more carbon-carbon double bonds, which may be branched or unbranched, substituted (mono- or poly-) or unsubstituted. Preferably the alkenyl group is a C₂₋₂₀ alkenyl group, more preferably a C₂₋₁₅ alkenyl group, more preferably still a C₂₋₁₂ alkenyl group, or preferably a C₂₋₄ alkenyl group, more preferably a C₂₋₃ alkenyl group. Suitable substituents include, for example, one or more R¹² or R^{12a} groups as defined above.

As used herein, the term “aryl” refers to a C₆₋₁₂ aromatic group which may be substituted (mono- or poly-) or unsubstituted. Typical examples include phenyl and naphthyl etc. Suitable substituents include, for example, one or more R¹² or R^{12a} groups.

As used herein, the term “heteroaryl” refers to a C₂₋₁₂ aromatic, substituted (mono- or poly-) or unsubstituted group, which comprises one or more heteroatoms. Preferably, the heteroaryl group is a C₄₋₁₂ aromatic group comprising one or more heteroatoms selected from N, O and S. Suitable heteroaryl groups include pyrrole, pyrazole, pyrimidine, pyrazine, pyridine, quinoline, thiophene, 1,2,3-triazole, 1,2,4-triazole, thiazole, oxazole, iso-thiazole, iso-oxazole, imidazole, furan and the like. Again, suitable substituents include, for example, one or more R¹² or R^{12a} groups.

As used herein, the term “heterocycloalkyl” refers to a cyclic aliphatic group which contains one or more heteroatoms. Preferred heterocycloalkyl groups include piperidinyl, pyrrolidinyl, piperazinyl and morpholinyl. More preferably, the heterocycloalkyl group is selected from N-piperidinyl, N-pyrrolidinyl, N-piperazinyl and N-morpholinyl

As used herein, the term “aralkyl” includes, but is not limited to, a group having both aryl and alkyl functionalities. By way of example, the term includes groups in which one of the hydrogen atoms of the alkyl group is replaced by an aryl group, e.g. a phenyl group optionally having one or more substituents such as halo, alkyl, alkoxy, hydroxy, and the like. Typical aralkyl groups include benzyl, phenethyl and the like.

As mentioned above, a first aspect of the invention relates to compounds of formula I,

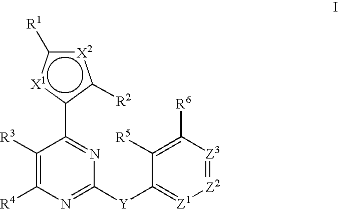

or pharmaceutically acceptable salts thereof, as defined above.

In one preferred embodiment, Y is NR¹¹. More preferably, Y is NH.

In one preferred embodiment, R³ and R⁴ are both H.

In one preferred embodiment, each R¹⁵ is independently selected from ethyl, ethyl, isopropyl, n-butyl, isobutyl, t-butyl, cyclopropyl, cyclobutyl, cyclopentyl, cyclohexyl, phenyl, pyridinyl, pyrrolidinyl, pyrrolyl, morpholinyl, piperazinyl, piperidinyl, triazolyl, tetrazolyl and thiazolyl.

In one preferred embodiment, each R¹² is independently selected from OH, OMe, COMe, CHO, CO₂Me, COOH, CN, CONH₂, NHMe, NH₂, NMe₂, SH, SMe, SOMe, SO₂Me, SO₂NHMe, SO₂NH₂, Cl, Br, F, I, CF₃, NO₂, N-morpholinyl, N-pyrrolidinyl and N-piperazinyl.

In one preferred embodiment,

R⁵, R⁶ and each R¹⁰ are each independently selected from H and (CH₂)ₘR¹²;
each R¹² is independently selected from R¹³, NR¹³COR¹⁴, NR¹³R¹⁴, SO₂R¹³, NR¹³SO₂R¹⁴, OR¹³, alkyl, NO₂, CF₃, alkoxy, halogen;
R¹³ and R¹⁴ are each independently H or (CH₂)ₙR¹⁵; and
each R¹⁵ is independently selected from alkyl, heteroaryl, aryl and heterocycloalkyl, each of which may be optionally substituted by one or more substituents selected from halogen, OH, CN, COO-alkyl, COOH, CO-alkyl, aralkyl, SO₂-alkyl, SO₂-aryl, CO-aryl, alkyl, alkoxy, NH₂, NH-alkyl, N(alkyl)₂ and CF₃.

In a more preferred embodiment,

R⁵, R⁶ and each R¹⁰ are each independently selected from H and R¹²;
each R¹² is independently selected from R¹³, NHCOR¹⁴, NR¹³R¹⁴, SO₂R¹³, NHSO₂R¹⁴, OR¹³, alkyl, NO₂, CF₃, alkoxy, halogen;
R¹³ and R¹⁴ are each independently H or R¹⁵; and
each R¹⁵ is independently selected from alkyl, aryl and heterocycloalkyl, each of which may be optionally substituted by one or more substituents selected from halogen, OH, CO-alkyl, aralkyl, SO₂-alkyl, SO₂-aryl, CO-aryl, alkyl, alkoxy, NH₂, NH-alkyl and N(alkyl)₂.

In one particularly preferred embodiment, R⁵, R⁶ and each R¹⁰ are each independently selected from H, Me, NO₂, CF₃, OMe, F, N-morpholinyl, N-piperazinyl and N-piperidinyl, said N-morpholinyl, N-piperazinyl and N-piperidinyl groups being optionally substituted by one or more substituents selected from halogen, OH, CN, COO-Me, COOH, CO-Me, CO-phenyl, Me, OMe, NH₂, NH-Me, NMe₂ and CF₃.

In one preferred embodiment, X¹ is CR⁸ and X² is NR⁷.

Preferably, R⁷ is H or Me.

In one preferred embodiment, R¹, R² and each R¹⁰ are each independently selected from H, CN, NO₂, alkyl, CONR¹³R¹⁴, NR¹³R¹⁴, NHCOR¹³OR¹³, R³, and NR¹³SO₂R¹⁴.

In a more preferred embodiment, R¹, R² and each R¹⁰ are each independently selected from H, CN, NO₂, alkyl, NR¹³R¹⁴, NR¹¹COR¹⁴ and OR¹³, where R¹³ and R¹⁴ are each independently H or alkyl.

Even more preferably,

### X¹ is CR¹;

### X² is NR⁷;

R² and R⁸ are both alkyl;
R¹ is selected from H, CN, NO₂, alkyl, CONR¹³R¹⁴, NR¹³R¹⁴, NHCOR¹³OR¹³, R¹³, and NR¹³SO₂R¹⁴.

In one highly preferred embodiment, R¹ is H or CN.

In one preferred embodiment, Z² is N and Z′ and Z³ are each independently CR¹⁰.

Preferably, R⁵, R⁶ and each R¹⁰ are each independently selected from H, halo, alkyl, alkoxy and heterocycloalkyl optionally substituted by one or more alkyl or acyl substituents.

Even more preferably, R⁵, R⁶ and each R¹⁰ are each independently selected from H, halo, alkyl, alkoxy, N-piperazinyl, N-morpholinyl and N-piperidinyl, wherein said N-piperazinyl, N-morpholinyl and N-piperidinyl groups are optionally substituted by one or more alkyl or acyl substituents.

In one especially preferred embodiment,

### R⁵ is H;

R⁶ is H or alkyl;

### Z¹ is CH;

### Z² is N;

Z³ is CR¹⁰ where R¹⁰ is independently H, alkoxy, halo, N-piperazinyl, and where said N-piperazinyl group is optionally substituted by an acyl group.

In one particularly preferred embodiment, said compound is selected from the following:

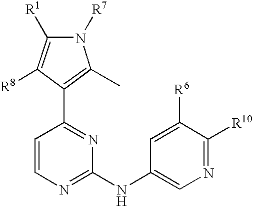

| Compound | R¹ | R⁶ | R⁷ | R⁸ | R¹⁰ |
|---|---|---|---|---|---|
| 26 | H | H | H | Me | OMe |
| 28 | CN | H | H | Me | OMe |
| 30 | CN | H | H | Me | Cl |
| 33 | CN | Me | H | Me | Cl |
| 38 | CN | H | H | Me | N—Ac Piperazine |
| 39 | CN | H | Me | Me | N—Ac Piperazine |

and pharmaceutically acceptable salts thereof.

A second aspect of the invention relates to compounds of formula II, or a pharmaceutically acceptable salt thereof,

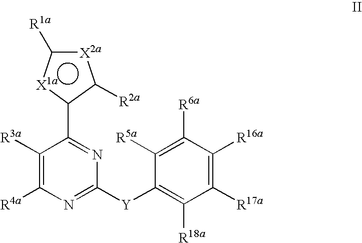

as defined above.

Preferably, Y is NR^{11a}, more preferably, NH.

In one preferred embodiment, R^{3a} and R^{4a} are both H.

In one preferred embodiment, each R^{15a} is independently selected from ethyl, ethyl, isopropyl, n-butyl, isobutyl, t-butyl, cyclopropyl, cyclobutyl, cyclopentyl, cyclohexyl, phenyl, pyridinyl, pyrrolidinyl, pyrrolyl, morpholinyl, piperazinyl, piperidinyl, triazolyl, tetrazolyl and thiazolyl.

In one preferred embodiment, each R^{12a} is independently selected from OH, OMe, COMe, CHO, CO₂Me, COOH, CN, CONH₂, NHMe, NH₂, NMe₂, SH, SMe, SOMe, SO₂Me, SO₂NHMe, SO₂NH₂, Cl, Br, F, I, CF₃, NO₂, N-morpholinyl, N-pyrrolidinyl and N-piperazinyl.

In one particularly preferred embodiment,

R^{5a}, R^{6a}, R^{16a}, R^{17a} and R^{18a} are each independently selected from H and (CH₂)ₘR^{12a};
each R^{12a} is independently selected from R^{13a}, NR^{13a}COR^{14a}, NR^{13a}R^{14a}, SO₂R^{13a}, NR^{13a}SO₂R^{14a}, OR^{13a}, alkyl, NO₂, CF₃, alkoxy, halogen and SO₂NR^{13a}R^{14a};
R^{13a} and R^{14a} are each independently H or (CH₂)ₙR^{15a}; and
each R^{15a} is independently selected from alkyl, heteroaryl, aryl and heterocycloalkyl, each of which may be optionally substituted by one or more substituents selected from halogen, OH, CN, COO-alkyl, COOH, CO-alkyl, aralkyl, SO₂-alkyl, SO₂-aryl, CO-aryl, alkyl, alkoxy, NH₂, NH-alkyl, N(alkyl)₂ and CF₃.

More preferably,

R^{5a}, R^{6a}, R^{16a}, R^{17a} and R^{18a} are each independently selected from H and R^{12a};
each R^{12a} is independently selected from R^{13a}, NHCOR^{14a}, NR^{13a}R^{14a}, SO₂R^{13a}, NHSO₂R^{14a}, OR^{13a}, alkyl, NO₂, CF₃, alkoxy, halogen and SO₂NR^{13a}R^{14a};
R^{13a} and R^{14a} are each independently H or R^{15a}; and
each R^{15a} is independently selected from alkyl, aryl and heterocycloalkyl, each of which may be optionally substituted by one or more substituents selected from halogen, OH, CO-alkyl, aralkyl, SO₂-alkyl, SO₂-aryl, CO-aryl, alkyl, alkoxy, NH₂, NH-alkyl and N(alkyl)₂.

Even more preferably, R^{5a}, R^{6a}, R^{6a}, R^{17a} and R^{18a} are each independently selected from H, Me, NO₂, CF₃, OMe, F, SO₂NH-alkyl, N-morpholinyl, N-piperazinyl and N-piperidinyl, said alkyl, N-morpholinyl, N-piperazinyl and N-piperidinyl groups being optionally substituted by one or more substituents selected from halogen, OH, CN, COO-Me, COOH, CO-Me, CO-phenyl, Me, OMe, NH₂, NH-Me, NMe₂ and CF₃.

In one preferred embodiment, X^{1a} is CR^{1a} and X^{2a} is NR^{7a}.

In one preferred embodiment, R^{7a} is H or Me.

In one preferred embodiment, R^{1a}, R^{2a} and R^{8a} are each independently selected from H, CN, NO₂, alkyl, CONR^{13a}R^{14a}, NR^{13a}R^{14a}, NHCOR^{13a}OR^{13a}, R^{13a}, and NR^{13a}SO₂R^{14a}.

In an even more preferred embodiment, R^{1a}, R^{2a} and R^{8a} are each independently selected from H, CN, NO₂, alkyl, NR^{13a}R^{14a}, NR^{13a}COR^{14a} and OR^{13a}, where R^{13a} and R^{14a} are each independently H or alkyl.

In one especially preferred embodiment,

### X^{1a} is CR^{8a};

### X^{2a} is NR^{7a};

R^{2a} is alkyl;
R^{8a} is alkyl or aryl;
R^{1a} is selected from H, CN, NO₂, COOR^{3a} alkyl, CONR^{13a}R^{14a}, NR^{13a}R^{14a}, NHCOR^{13a} OR^{13a}, R^{13a}, and NR^{13a}SO₂R^{14a}.

Even more preferably, R^{2a} and R^{8a} are both methyl.

In one preferred embodiment, R^{1a} is H, CN, CO₂(CH₂)₂OMe or CO₂Et.

In one preferred embodiment, R^{5a}, R^{6a}, R^{16a}, R^{17a} and R^{18a} are each independently selected from H, halo, SO₂NR^{13a}R^{14a}, alkoxy and heterocycloalkyl optionally substituted by one or more alkyl, aralkyl or acyl substituents.

In a more preferred embodiment, R^{5a}, R^{6a}, R^{16a}, R^{17a} and R^{18a} are each independently selected from H, halo, SO₂NR^{13a}R^{14a}, alkoxy, N-piperazinyl, N-morpholinyl and N-piperidinyl, wherein said N-piperazinyl, N-morpholinyl and N-piperidinyl groups are optionally substituted by one or more alkyl, aralkyl or acyl substituents, and wherein R^{13a} and R^{14a} are each independently alkyl groups optionally substituted by one or more OH or alkoxy groups.

In a further preferred embodiment, R^{5a}, R^{6a}, R^{16a}, R^{17a} and R^{18a} are each independently selected from H, halo, alkoxy, SO₂NHEt, SO₂NHⁱPr, SO₂NHC(Me)₂CH₂OH, SO₂NHCH₂CH₂OMe, N-morpholinyl, N-Ac-piperazinyl, N-Bz-piperazinyl and bis-methyl-morpholinyl.

In one highly preferred embodiment,

### R^{5a} and R^{18a} are H;

R^{6a} is H or halo;
R^{16a} and R^{17a} are each independently selected from H, halo, alkoxy, SO₂NHEt, SO₂NHⁱPr, SO₂NHC(Me)₂CH₂OH, SO₂NHCH₂CH₂OMe, N-morpholinyl, N-Ac-piperazinyl, N-Bz-piperazinyl and bis-methyl-morpholinyl.

In a particularly preferred embodiment, the compound of formula II is selected from the following:

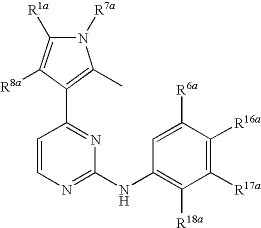

| Compound | R^{1a} | R^{7a} | R^{8a} | R^{6a} | R^{16a} | R^{17a} | R^{18a} |
|---|---|---|---|---|---|---|---|
| 4 | H | H | Me | H | Morph | H | H |
| 24 | CN | H | Me | H | Morph | H | H |
| 32 | CN | H | Me | H | H | 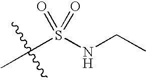 | H |
| 34 | CN | H | Me | H | H | 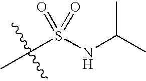 | H |
| 36 | CN | H | Me | H | 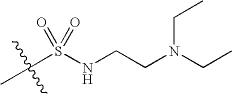 | H | H |
| 37 | CO₂(CH₂)₂OMe | H | Ph | H | Morph | H | H |
| 40 | CN | H | Me | H | N-Bz | H | H |
| | | | | | Piperizine | | |
| 41 | CN | H | Me | H | N—Ac | H | H |
| | | | | | Piperizine | | |
| 42 | H | H | Ph | H | Morph | H | H |
| 43 | CO₂Et | H | Me | H | Morph | H | H |
| 44 | H | H | Me | H | Morph | Cl | H |
| 45 | H | H | Me | Cl | Morph | Cl | H |
| 46 | CO₂Et | H | Me | H | Morph | Cl | H |
| 47 | CO₂Et | H | Me | Cl | Morph | Cl | H |
| 48 | H | Me | Me | H | Morph | H | H |
| 49 | H | Me | Me | H | Morph | Cl | H |
| 50 | H | H | Me | H | Morph | H | Me |
| 51 | H | H | Me | H | bis-methyl | H | H |
| | | | | | Morph | | |
| 54 | CO₂Et | H | Me | H | Morph | OMe | H |
| 59 | H | H | Me | H | Morph | OMe | H |
| 60 | H | H | Me | H | N—Ac- | H | H |
| | | | | | piperazine | | |

and pharmaceutically acceptable salts thereof

A further aspect of the invention relates to a compound selected from the following:

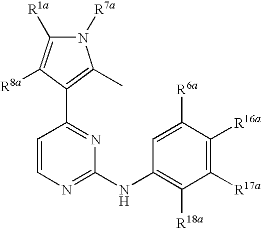

| Compound | R^{1a} | R^{7a} | R^{8a} | R^{6a} | R^{16a} | R^{17a} | R^{18a} |
|---|---|---|---|---|---|---|---|
| 1 | H | H | Me | H | CN | H | H |
| 2 | H | H | Me | H | H | CN | H |
| 3 | H | H | Me | H | H | CH₂OH | H |
| 5 | CO₂Et | H | Ph | H | H | NO₂ | H |
| 6 | CO₂Et | H | Ph | H | OH | H | H |
| 7 | CO₂Et | H | Ph | H | H | OH | H |
| 8 | CO₂Et | H | Ph | H | H | F | H |
| 9 | CO₂Et | H | Ph | H | I | H | H |
| 10 | CO₂Et | H | Ph | H | CF₃ | H | H |
| 11 | CO₂Et | H | Ph | H | OH | NO₂ | H |
| 12 | CO₂Et | H | Ph | H | NMe₂ | H | H |
| 13 | H | H | Ph | H | H | NO₂ | H |
| 14 | H | H | Ph | H | I | H | H |
| 15 | H | H | Ph | H | OH | H | H |
| 16 | H | H | Ph | H | F | H | H |
| 17 | CH₂NMe₂ | H | Me | H | H | NO₂ | H |
| 18 | CN | H | Me | H | NMe₂ | NO₂ | H |
| 19 | NO₂ | H | Me | H | NMe₂ | NO₂ | H |
| 20 | CN | Me | Me | H | NMe₂ | H | H |
| 22 | CO₂Et | H | Ph | H | F | H | H |
| 27 | CN | H | Me | NO₂ | H | H | Me |
| 29 | CN | H | Me | OMe | H | H | Me |
| 31 | CN | H | Me | H | NHMe | CF₃ | H |
| 52 | CN | H | Me | OMe | OMe | OMe | H |
| 53 | CN | Me | Me | OMe | OMe | OMe | H |
| 55 | CO₂Et | H | Me | OMe | H | OMe | H |
| 56 | H | H | Me | OMe | OMe | OMe | H |
| 57 | H | Me | Me | OMe | OMe | OMe | H |
| 58 | CO₂Et | H | Me | OMe | OMe | OMe | H |

and pharmaceutically acceptable salts thereof.

In one preferred embodiment, the compound of the invention is capable of inhibiting one or more protein kinases, as measured by the assays described in the accompanying Examples section. Preferably, the compound of the invention exhibits a Ki value of less than 10 μM, more preferably less than 5 μM, even more preferably less than 1 μM or less than 0.5 less μM, more preferably still less than 0.1 μM. More preferably still, the compound exhibits a Ki value of less than 0.01 μM, even more preferably less than 0.005 μM.

In one highly preferred embodiment of the invention, the compound is capable of preferentially inhibiting GSK3 over one or more other protein kinases; thus the compound is capable of selectively inhibiting GSK3 over one or more other protein kinases. As used herein, the term “selectively” refers to the compounds that are selective for GSK over one or more other protein kinases. Preferably, the selectivity ratio for GSK over one or more other protein kinases is greater than about 2 to 1, more preferably greater than about 5 to 1 or about 10 to 1, even more preferably greater than about 100 to 1. Selectivity ratios may be determined by the skilled person in the art.

In one highly preferred embodiment of the invention, the compound is capable of preferentially inhibiting Aurora-A over one or more other protein kinases; thus the compound is capable of selectively inhibiting Aurora-A over one or more other protein kinases. As used herein, the term “selectively” refers to the compounds that are selective for Aurora-A over one or more other protein kinases. Preferably, the selectivity ratio for Aurora-A over one or more other protein kinases is greater than about 2 to 1, more preferably greater than about 5 to 1 or about 10 to 1, even more preferably greater than about 100 to 1. Selectivity ratios may be determined by the skilled person in the art.

### Therapeutic Use

The compounds of the invention have been found to possess anti-proliferative activity and are therefore believed to be of use in the treatment of proliferative disorders such as cancers, leukaemias and other disorders associated with uncontrolled cellular proliferation such as psoriasis and restenosis.

Thus, one aspect of the invention relates to the use of a compound of the invention, or a pharmaceutically acceptable salt thereof, in the preparation of a medicament for treating a proliferative disorder.

As used herein the phrase “preparation of a medicament” includes the use of one or more of the above described compounds directly as the medicament in addition to its use in a screening programme for further anti-viral and/or antiproliferative agents or in any stage of the manufacture of such a medicament.

As defined herein, an anti-proliferative effect within the scope of the present invention may be demonstrated by the ability to inhibit cell proliferation in an in vitro whole cell assay, for example using any of the cell lines AGS, H1299 or SJSA-1, or by showing inhibition of the interaction between HDM2 and p53 in an appropriate assay. These assays, including methods for their performance, are described in more detail in the accompanying Examples. Using such assays it may be determined whether a compound is anti-proliferative in the context of the present invention.

One preferred embodiment therefore relates to the use of one or more compounds of the invention in the treatment of proliferative disorders. Preferably, the proliferative disorder is a cancer or leukaemia. The term proliferative disorder is used herein in a broad sense to include any disorder that requires control of the cell cycle, for example cardiovascular disorders such as restenosis and cardiomyopathy, auto-immune disorders such as glomerulonephritis and rheumatoid arthritis, dermatological disorders such as psoriasis, anti-inflammatory, anti-fungal, antiparasitic disorders such as malaria, emphysema and alopecia. In these disorders, the compounds of the present invention may induce apoptosis or maintain stasis within the desired cells as required.

In one preferred embodiment, the proliferative disorder is cancer or leukaemia.

In another preferred embodiment, the proliferative disorder is glomerulonephritis, rheumatoid arthritis, psoriasis or chronic obstructive pulmonary disorder.

The compounds of the invention may inhibit any of the steps or stages in the cell cycle, for example, formation of the nuclear envelope, exit from the quiescent phase of the cell cycle (G0), G1 progression, chromosome decondensation, nuclear envelope breakdown, START, initiation of DNA replication, progression of DNA replication, termination of DNA replication, centrosome duplication, G2 progression, activation of mitotic or meiotic functions, chromosome condensation, centrosome separation, microtubule nucleation, spindle formation and function, interactions with microtubule motor proteins, chromatid separation and segregation, inactivation of mitotic functions, formation of contractile ring, and cytokinesis functions. In particular, the compounds of the invention may influence certain gene functions such as chromatin binding, formation of replication complexes, replication licensing, phosphorylation or other secondary modification activity, proteolytic degradation, microtubule binding, actin binding, septin binding, microtubule organising centre nucleation activity and binding to components of cell cycle signalling pathways.

In one embodiment, the compound of the invention is administered in an amount sufficient to inhibit at least one CDK enzyme. Assays for determining CDK activity are described in more detail in the accompanying examples.

A further aspect of the invention relates to a method of treating a CDK-dependent disorder, said method comprising administering to a subject in need thereof, a compound of the invention or a pharmaceutically acceptable salt thereof, as defined above in an amount sufficient to inhibit a CDK.

Another aspect relates to the use of a compound of the invention as an anti-mitotic agent.

Another aspect of the invention relates to the use of a compound of the invention as an antiviral agent.

Thus, another aspect of the invention relates to the use of a compound of the invention in the preparation of a medicament for treating a viral disorder, such as human cytomegalovirus (HCMV), herpes simplex virus type 1 (HSV-1), human immunodeficiency virus type 1 (HIV-1), and varicella zoster virus (VZV).

In a more preferred embodiment of the invention, the compound of the invention is administered in an amount sufficient to inhibit one or more of the host cell CDKs involved in viral replication, i.e. CDK2, CDK7, CDK8, and CDK9 [Wang D, De la Fuente C, Deng L, Wang L, Zilberman I, Eadie C, Healey M, Stein D, Denny T, Harrison L E, Meijer L, Kashanchi F., Inhibition of human immunodeficiency virus type 1 transcription by chemical cyclin-dependent kinase inhibitors, 3. Virol. 2001; 75: 7266-7279].

As defined herein, an anti-viral effect within the scope of the present invention may be demonstrated by the ability to inhibit CDK2, CDK7, CDK8 or CDK9.

In a particularly preferred embodiment, the invention relates to the use of one or more compounds of the invention in the treatment of a viral disorder which is CDK dependent or sensitive. CDK dependent disorders are associated with an above normal level of activity of one or more CDK enzymes. Such disorders preferably associated with an abnormal level of activity of CDK2, CDK7, CDK8 and/or CDK9. A CDK sensitive disorder is a disorder in which an aberration in the CDK level is not the primary cause, but is downstream of the primary metabolic aberration. In such scenarios, CDK2, CDK7, CDK8 and/or CDK9 can be said to be part of the sensitive metabolic pathway and CDK inhibitors may therefore be active in treating such disorders.

Another aspect relates to the use of compounds of the invention, or pharmaceutically acceptable salts thereof, in the preparation of a medicament for treating diabetes.

In a particularly preferred embodiment, the diabetes is type II diabetes.

GSK3 is one of several protein kinases that phosphorylate glycogen synthase (GS). The stimulation of glycogen synthesis by insulin in skeletal muscle results from the dephosphorylation and activation of GS. GSK3's action on GS thus results in deactivation of the latter and thus suppression of the conversion of glucose into glycogen in muscles.

Type II diabetes (non-insulin dependent diabetes mellitus) is a multi-factorial disease. Hyperglycaemia is due to insulin resistance in the liver, muscles, and other tissues, coupled with impaired secretion of insulin. Skeletal muscle is the main site for insulin-stimulated glucose uptake, there it is either removed from circulation or converted to glycogen. Muscle glycogen deposition is the main determinant in glucose homeostasis and type II diabetics have defective muscle glycogen storage. There is evidence that an increase in GSK3 activity is important in type II diabetes [Chen, Y. H.; Hansen, L.; Chen, M. X.; Bjorbaek, C.; Vestergaard, H.; Hansen, T.; Cohen, P. T.; Pedersen, O. Diabetes, 1994, 43, 1234]. Furthermore, it has been demonstrated that GSK3 is over-expressed in muscle cells of type II diabetics and that an inverse correlation exists between skeletal muscle GSK3 activity and insulin action [Nikoulina, S. E.; Ciaraldi, T. P.; Mudaliar, S.; Mohideen, P.; Carter, L.; Henry, R. R. Diabetes, 2000, 49, 263].

GSK3 inhibition is therefore of therapeutic significance in the treatment of diabetes, particularly type II, and diabetic neuropathy.

It is notable that GSK3 is known to phosphorylate many substrates other than GS, and is thus involved in the regulation of multiple biochemical pathways. For example, GSK is highly expressed in the central and peripheral nervous systems.

Another aspect therefore relates to the use of compounds of the invention, or pharmaceutically acceptable salts thereof, in the preparation of a medicament for treating a CNS disorders, for example neurodegenerative disorders.

In one preferred embodiment, the neurodegenerative disorder is neuronal apoptosis.

In another preferred embodiment, the CNS disorder is Alzheimer's disease.

Tau is a GSK-3 substrate which has been implicated in the etiology of Alzheimer's disease. In healthy nerve cells, Tau co-assembles with tubulin into microtubules. However, in Alzheimer's disease, tau forms large tangles of filaments, which disrupt the microtubule structures in the nerve cell, thereby impairing the transport of nutrients as well as the transmission of neuronal messages.

Without wishing to be bound by theory, it is believed that GSK3 inhibitors may be able to prevent and/or reverse the abnormal hyperphosphorylation of the microtubule-associated protein tau that is an invariant feature of Alzheimer's disease and a number of other neurodegenerative diseases, such as progressive supranuclear palsy, corticobasal degeneration and Pick's disease. Mutations in the tau gene cause inherited forms of fronto-temporal dementia, further underscoring the relevance of tau protein dysfunction for the neurodegenerative process [Goedert, M. Curr. Opin. Gen. Dev., 2001, 11, 343].

Another aspect relates to the use of compounds of the invention, or pharmaceutically acceptable salts thereof, in the preparation of a medicament for treating bipolar disorder.

Yet another aspect relates to the use of compounds of the invention, or pharmaceutically acceptable salts thereof, in the preparation of a medicament for treating a stroke.

Reducing neuronal apoptosis is an important therapeutic goal in the context of head trauma, stroke, epilepsy, and motor neuron disease [Mattson, M. P. Nat. Rev. Mol. Cell. Biol., 2000, 1, 120]. Therefore, GSK3 as a pro-apoptotic factor in neuronal cells makes this protein kinase an attractive therapeutic target for the design of inhibitory drugs to treat these diseases.

Yet another aspect relates to the use of compounds of the invention, or pharmaceutically acceptable salts thereof, in the preparation of a medicament for treating alopecia.

Hair growth is controlled by the Wnt signalling pathway, in particular Wnt-3. In tissue-culture model systems of the skin, the expression of non-degradable mutants of β-catenin leads to a dramatic increase in the population of putative stem cells, which have greater proliferative potential [Zhu, A. J.; Watt, F. M. Development, 1999, 126, 2285]. This population of stem cells expresses a higher level of non-cadherin-associated β-catenin [DasGupta, R; Fuchs, E. Development, 1999, 126, 4557], which may contribute to their high proliferative potential. Moreover, transgenic mice overexpressing a truncated β-catenin in the skin undergo de novo hair-follicle morphogenesis, which normally is only established during embryogenesis. The ectopic application of GSK3 inhibitors may therefore be therapeutically useful in the treatment of baldness and in restoring hair growth following chemotherapy-induced alopecia.

A further aspect of the invention relates to a method of treating a GSK3-dependent disorder, said method comprising administering to a subject in need thereof, a compound of the invention or a pharmaceutically acceptable salt thereof, as defined above in an amount sufficient to inhibit GSK3.

Preferably, the compound of the invention, or pharmaceutically acceptable salt thereof, is administered in an amount sufficient to inhibit GSK3β.

In one embodiment of the invention, the compound of the invention is administered in an amount sufficient to inhibit at least one PLK enzyme.

A further aspect of the invention relates to a method of treating a PLK-dependent disorder, said method comprising administering to a subject in need thereof, a compound of the invention or a pharmaceutically acceptable salt thereof, as defined above in an amount sufficient to inhibit PLK.

The polo-like kinases (PLKs) constitute a family of serine/threonine protein kinases. Mitotic Drosophila melanogaster mutants at the polo locus display spindle abnormalities [Sunkel et al., J. Cell Sci., 1988, 89, 25] and polo was found to encode a mitotic kinase [Llamazares et al., Genes Dev., 1991, 5, 2153]. In humans, there exist three closely related PLKs [Glover et al., Genes Dev., 1998, 12, 3777]. They contain a highly homologous amino-terminal catalytic kinase domain and their carboxyl termini contain two or three conserved regions, the polo boxes. The function of the polo boxes remains incompletely understood but they are implicated in the targeting of PLKs to subcellular compartments [Lee et al., Proc. Natl. Acad. Sci. USA, 1998, 95, 9301; Leung et al., Nat. Struct. Biol., 2002, 9, 719], mediation of interactions with other proteins [Kauselmann et al., EMBO J., 1999, 18, 5528], or may constitute part of an autoregulatory domain [Nigg, Curr. Opin. Cell Biol., 1998, 10, 776]. Furthermore, the polo box-dependent PLK1 activity is required for proper metaphase/anaphase transition and cytokinesis [Yuan et al., Cancer Res., 2002, 62, 4186; Seong et al., J. Biol. Chem., 2002, 277, 32282].

Studies have shown that human PLKs regulate some fundamental aspects of mitosis [Lane et al., J. Cell. Biol., 1996, 135, 1701; Cogswell et al., Cell Growth Differ., 2000, 11, 615]. In particular, PLK1 activity is believed to be necessary for the functional maturation of centrosomes in late G2/early prophase and subsequent establishment of a bipolar spindle. Depletion of cellular PLK1 through the small interfering RNA (siRNA) technique has also confirmed that this protein is required for multiple mitotic processes and completion of cytokinesis [Liu et al., Proc. Natl. Acad. Sci. USA, 2002, 99, 8672].

In a more preferred embodiment of the invention, the compound of the invention is administered in an amount sufficient to inhibit PLK1.

Of the three human PLKs, PLK1 is the best characterized; it regulates a number of cell division cycle effects, including the onset of mitosis [Toyoshima-Morimoto et al., Nature, 2001, 410, 215; Roshak et al., Cell. Signalling, 2000, 12, 405], DNA-damage checkpoint activation [Smits et al., Nat. Cell Biol., 2000, 2, 672; van Vugt et al., J. Biol. Chem., 2001, 276, 41656], regulation of the anaphase promoting complex [Sumara et al., Mol. Cell, 2002, 9, 515; Golan et al., J. Biol. Chem., 2002, 277, 15552; Kotani et al., Mol. Cell, 1998, 1, 371], phosphorylation of the proteasome [Feng et al., Cell Growth Differ., 2001, 12, 29], and centrosome duplication and maturation [Dai et al., Oncogene, 2002, 21, 6195].

Specifically, initiation of mitosis requires activation of M-phase promoting factor (MPF), the complex between the cyclin dependent kinase CDK1 and B-type cyclins [Nurse, Nature, 1990, 344, 503]. The latter accumulate during the S and G2 phases of the cell cycle and promote the inhibitory phosphorylation of the MPF complex by WEEI, MIK1, and MYTI kinases. At the end of the G2 phase, corresponding dephosphorylation by the dual-specificity phosphatase CDC25C triggers the activation of MPF [Nigg, Nat. Rev. Mol. Cell. Biol., 2001, 2, 21]. In interphase, cyclin B localizes to the cytoplasm [Hagting et al., EMBO J., 1998, 17, 4127], it then becomes phosphorylated during prophase and this event causes nuclear translocation [Hagting et al., Curr. Biol., 1999, 9, 680; Yang et al., J. Biol. Chem., 2001, 276, 3604]. The nuclear accumulation of active MPF during prophase is thought to be important for initiating M-phase events [Takizawa et al, Curr. Opin. Cell Biol., 2000, 12, 658]. However, nuclear MPF is kept inactive by WEE1 unless counteracted by CDC25C. The phosphatase CDC25C itself, localized to the cytoplasm during interphase, accumulates in the nucleus in prophase [Seki et al, Mol. Biol. Cell, 1992, 3, 1373; Heald et al, Cell, 1993, 74, 463; Dalal et al., Mol. Cell. Biol., 1999, 19, 4465). The nuclear entry of both cyclin B [Toyoshima-Morimoto et al., Nature, 2001, 410, 215] and CDC25C [Toyoshima-Morimoto et al., EMBO Rep., 2002, 3, 341] are promoted through phosphorylation by PLK1 [Roshak et al., Cell. Signalling, 2000, 12, 405]. This kinase is an important regulator of M-phase initiation.

In one particularly preferred embodiment, the compounds of the invention are ATP-antagonistic inhibitors of PLK1.

In the present context ATP antagonism refers to the ability of an inhibitor compound to diminish or prevent PLK catalytic activity, i.e. phosphotransfer from ATP to a macromolecular PLK substrate, by virtue of reversibly or irreversibly binding at the enzyme's active site in such a manner as to impair or abolish ATP binding.

In another preferred embodiment, the compound of the invention is administered in an amount sufficient to inhibit PLK2 and/or PLK3.

Mammalian PLK2 (also known as SNK) and PLK3 (also known as PRK and FNK) were originally shown to be immediate early gene products. PLK3 kinase activity appears to peak during late S and G2 phase. It is also activated during DNA damage checkpoint activation and severe oxidative stress. PLK3 also plays an important role in the regulation of microtubule dynamics and centrosome function in the cell and deregulated PLK3 expression results in cell cycle arrest and apoptosis [Wang et al., Mol. Cell. Biol., 2002, 22, 3450]. PLK2 is the least well understood homologue of the three PLKs. Both PLK2 and PLK3 may have additional important post-mitotic functions [Kauselmann et al., EMBO J., 1999, 18, 5528].

In another preferred embodiment, the compound of the invention is administered in an amount sufficient to inhibit at least one aurora kinase. Preferably, the aurora kinase is aurora kinase A, aurora kinase B or aurora kinase C.

A further aspect of the invention relates to a method of treating an aurora kinase-dependent disorder, said method comprising administering to a subject in need thereof, a compound of the invention or a pharmaceutically acceptable salt thereof, as defined above in an amount sufficient to inhibit an aurora kinase.

In another preferred embodiment, the compound of the invention is administered in an amount sufficient to inhibit at least one tyrosine kinase.

Preferably, the tyrosine kinase is Ableson tyrosine kinase (BCR-ABL), FMS-related tyrosine kinase 3 (FLT3), platelet-derived growth factor (PDGF) receptor tyrosine kinase or vascular endothelial growth factor (VEGF) receptor tyrosine kinase.

A further aspect of the invention relates to a method of treating a tyrosine kinase-dependent disorder, said method comprising administering to a subject in need thereof, a compound of the invention or a pharmaceutically acceptable salt thereof as defined above in an amount sufficient to inhibit a tyrosine kinase.

Another aspect relates to the use of a compound of the invention for inhibiting a protein kinase.

A further aspect of the invention relates to a method of inhibiting a protein kinase, said method comprising contacting said protein kinase with a compound of the invention.

Preferably, the protein kinase is selected from a CDK, GSK, an aurora kinase, PLK and a tyrosine kinase.

In a preferred embodiment of this aspect, the protein kinase is a cyclin dependent kinase. Preferably, the protein kinase is CDK1, CDK2, CDK3, CDK4, CDK6, CDK7, CDK8 or CDK9, more preferably CDK2.

The compounds of the invention are also useful in the preparation of medicaments for the treatment of various ophthalmic disorders. Preferably, the ophthalmic disorder is glaucoma, exudative age-related macular degeneration (AMD) or proliferative diabetic retinopathy (PDR).

The disease state referred to as glaucoma is characterized by a permanent loss of visual function due to irreversible damage to the optic nerve. The several morphologically or functionally distinct types of glaucoma are typically characterized by elevated intraocular pressure (IOP), which is considered to be causally related to the pathological course of the disease. Ocular hypertension is a condition wherein intraocular pressure is elevated, but no apparent loss of visual function has occurred; such patients are considered to be a high risk for the eventual development of the visual loss associated with glaucoma. GSK-3 inhibitors are useful for the treatment of eye diseases such as glaucoma. It has been shown that a component of the Wnt signalling pathway, frizzled related protein (FRP), is differentially expressed in a number of glaucomatous trabecular meshwork cell lines and can disrupt the normal signalling cascade causing an increase in outflow resistance and development of elevated IOP. Hellberg M. R et al (US20040186159) have shown that through the interaction of GSK-3 with components of the Wnt signalling pathway, inhibition of GSK-3 by pharmacological agents can circumvent the FRP mediated antagonism of the Wnt signaling pathway caused by the elevated levels of FRP and counteract the increase in outflow resistance that results from the increase in production of FRP in individuals with glaucoma.

CTGF is a secreted cytokine which is known to increase extracellular matrix (ECM) production, primarily via increased deposition of collagen I and of fibronectin. Overexpression of CTGF has previously been implicated as a major causative factor in conditions such as scleroderma, fibroproliferative diseases, scarring, etc. in which there is an overaccumulation of ECM components. An overaccumulation of extracellular matrix materials in the region of the trabecular meshwork (TM) is also a hallmark of many forms of glaucoma; such increases are believed to lead to increased resistance to aqueous outflow, and therefore elevated intraocular pressures. Fleenor D L et al (US20050234075) have shown that GSK-3 inhibitors and CDK inhibitors can inhibit both basal and TGF.beta.2-induced CTGF expression in human TM cells therefore compounds of the current invention are useful for the treatment of glaucoma.

The compounds of the invention are also useful in the treatment of AMD and PDR. Exudative age-related macular degeneration (AMD) and proliferative diabetic retinopathy (PDR) are the major causes of acquired blindness in developed countries and are characterized by pathologic posterior segment neovascularization in the eye. The inciting cause in both exudative AMD and PDR is still unknown, however, the elaboration of various proangiogenic growth factors appears to be a common stimulus. Soluble growth factors, such as vascular endothelial growth factor (VEGF), platelet-derived growth factor (PDGF), basic fibroblast growth factor (bFGF or FGF-2), insulin-like growth factor 1 (IGF-1), angiopoietins, etc., have been found in tissues and fluids removed from patients with pathologic ocular angiogenesis. Inhibition or blockade of the activity of these growth factors and of other intracellular enzymes such as aurora kinases has been shown to have an antiangiogenic effect. Thus compounds of the current invention are useful for treating ophthalmic diseases characterised by neovascularization.

### Pharmaceutical Compositions

A further aspect of the invention relates to a pharmaceutical composition comprising a compound of the invention admixed with one or more pharmaceutically acceptable diluents, excipients or carriers. Even though the compounds of the present invention (including their pharmaceutically acceptable salts, esters and pharmaceutically acceptable solvates) can be administered alone, they will generally be administered in admixture with a pharmaceutical carrier, excipient or diluent, particularly for human therapy. The pharmaceutical compositions may be for human or animal usage in human and veterinary medicine.

Examples of such suitable excipients for the various different forms of pharmaceutical compositions described herein may be found in the “Handbook of Pharmaceutical Excipients, 2 Edition, (1994), Edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s).

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

### Salts/Esters

The compounds of the invention can be present as salts or esters, in particular pharmaceutically acceptable salts or esters.

Pharmaceutically acceptable salts of the compounds of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. sulphuric acid, phosphoric acid or hydrohalic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

### Enantiomers/Tautomers

In all aspects of the present invention previously discussed, the invention includes, where appropriate all enantiomers and tautomers of the compounds of the invention. The person skilled in the art will recognise compounds that possess an optical properties (one or more chiral carbon atoms) or tautomeric characteristics. The corresponding enantiomers and/or tautomers may be isolated/prepared by methods known in the art.

### Stereo and Geometric Isomers

Some of the compounds of the invention may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those inhibitor agents, and mixtures thereof. The terms used in the claims encompass these forms, provided said forms retain the appropriate functional activity (though not necessarily to the same degree).

The present invention also includes all suitable isotopic variations of the agent or a pharmaceutically acceptable salt thereof. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agent of the present invention and pharmaceutically acceptable salts thereof of this invention can generally be prepared by conventional procedures using appropriate isotopic variations of suitable reagents.

### Solvates

The present invention also includes solvate forms of the compounds of the present invention. The terms used in the claims encompass these forms.

### Polymorphs

The invention furthermore relates to the compounds of the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

### Prodrugs

The invention further includes the compounds of the present invention in prodrug form. Such prodrugs are generally compounds of the invention wherein one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. Examples of such modifications include ester (for example, any of those described above), wherein the reversion may be carried out be an esterase etc. Other such systems will be well known to those skilled in the art.

### Administration

The pharmaceutical compositions of the present invention may be adapted for oral, rectal, vaginal, parenteral, intramuscular, intraperitoneal, intraarterial, intrathecal, intrabronchial, subcutaneous, intradermal, intravenous, nasal, buccal or sublingual routes of administration.

For oral administration, particular use is made of compressed tablets, pills, tablets, gellules, drops, and capsules. Preferably, these compositions contain from 1 to 250 mg and more preferably from 10-100 mg, of active ingredient per dose.

Other forms of administration comprise solutions or emulsions which may be injected intravenously, intraarterially, intrathecally, subcutaneously, intradermally, intraperitoneally or intramuscularly, and which are prepared from sterile or sterilisable solutions. The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

Injectable forms may contain between 10-1000 mg, preferably between 10-250 mg, of active ingredient per dose.

Compositions may be formulated in unit dosage form, i.e., in the form of discrete portions containing a unit dose, or a multiple or sub-unit of a unit dose.

### Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

Depending upon the need, the agent may be administered at a dose of from 0.01 to 30 mg/kg body weight, such as from 0.1 to 10 mg/kg, more preferably from 0.1 to 1 mg/kg body weight.

In an exemplary embodiment, one or more doses of 10 to 150 mg/day will be administered to the patient for the treatment of malignancy.

### Combinations

In a particularly preferred embodiment, the one or more compounds of the invention are administered in combination with one or more other active agents, for example, existing anticancer drugs available on the market. In such cases, the compounds of the invention may be administered consecutively, simultaneously or sequentially with the one or more other active agents.

Anticancer drugs in general are more effective when used in combination. In particular, combination therapy is desirable in order to avoid an overlap of major toxicities, mechanism of action and resistance mechanism(s). Furthermore, it is also desirable to administer most drugs at their maximum tolerated doses with minimum time intervals between such doses. The major advantages of combining chemotherapeutic drugs are that it may promote additive or possible synergistic effects through biochemical interactions and also may decrease the emergence of resistance in early tumor cells which would have been otherwise responsive to initial chemotherapy with a single agent. An example of the use of biochemical interactions in selecting drug combinations is demonstrated by the administration of leucovorin to increase the binding of an active intracellular metabolite of 5-fluorouracil to its target, thymidylate synthase, thus increasing its cytotoxic effects.

Numerous combinations are used in current treatments of cancer and leukemia. A more extensive review of medical practices may be found in “Oncologic Therapies” edited by E. E. Vokes and H. M. Golomb, published by Springer.

Beneficial combinations may be suggested by studying the growth inhibitory activity of the test compounds with agents known or suspected of being valuable in the treatment of a particular cancer initially or cell lines derived from that cancer. This procedure can also be used to determine the order of administration of the agents, i.e. before, simultaneously, or after delivery. Such scheduling may be a feature of all the cycle acting agents identified herein.

### Assays

Another aspect of the invention relates to the use of a compound of the invention as defined hereinabove in an assay for identifying further candidate compounds that influence the activity of one or more of the following: a CDK, FLT-3, an aurora kinase, GSK-3, PLK and/or a tyrosine kinase.

Preferably, the assay is capable of identifying candidate compounds that are capable of inhibiting one or more of a CDK enzyme, FLT-3, an auroroa kinase, a tyrosine kinase, GSK or a PLK enzyme.

More preferably, the assay is a competitive binding assay.

Preferably, the candidate compound is generated by conventional SAR modification of a compound of the invention.

As used herein, the term “conventional SAR modification” refers to standard methods known in the art for varying a given compound by way of chemical derivatisation.

Thus, in one aspect, the identified compound may act as a model (for example, a template) for the development of other compounds. The compounds employed in such a test may be free in solution, affixed to a solid support, borne on a cell surface, or located intracellularly. The abolition of activity or the formation of binding complexes between the compound and the agent being tested may be measured.

The assay of the present invention may be a screen, whereby a number of agents are tested. In one aspect, the assay method of the present invention is a high through-put screen.

This invention also contemplates the use of competitive drug screening assays in which neutralising antibodies capable of binding a compound specifically compete with a test compound for binding to a compound.

Another technique for screening provides for high throughput screening (HTS) of agents having suitable binding affinity to the substances and is based upon the method described in detail in WO 84/03564.

It is expected that the assay methods of the present invention will be suitable for both small and large-scale screening of test compounds as well as in quantitative assays.

Preferably, the competitive binding assay comprises contacting a compound of the invention with a CDK, FLT-3, an aurora kinase, GSK-3, PLK and/or a tyrosine kinase enzyme in the presence of a known substrate of said enzyme and detecting any change in the interaction between said enzyme and said known substrate.

A further aspect of the invention provides a method of detecting the binding of a ligand to a CDK, FLT-3, an aurora kinase, GSK-3, PLK or a tyrosine kinase enzyme, said method comprising the steps of:
(i) contacting a ligand with a CDK, FLT-3, an aurora kinase, GSK-3, PLK or a tyrosine kinase enzyme in the presence of a known substrate of said enzyme;
(ii) detecting any change in the interaction between said enzyme and said known substrate;
   and wherein said ligand is a compound of the invention.

One aspect of the invention relates to a process comprising the steps of:
(a) performing an assay method described hereinabove;
(b) identifying one or more ligands capable of binding to a ligand binding domain; and
(c) preparing a quantity of said one or more ligands.

Another aspect of the invention provides a process comprising the steps of:
(a) performing an assay method described hereinabove;
(b) identifying one or more ligands capable of binding to a ligand binding domain; and
(c) preparing a pharmaceutical composition comprising said one or more ligands.

Another aspect of the invention provides a process comprising the steps of:
(a) performing an assay method described hereinabove;
(b) identifying one or more ligands capable of binding to a ligand binding domain;
(c) modifying said one or more ligands capable of binding to a ligand binding domain;
(d) performing the assay method described hereinabove;
(e) optionally preparing a pharmaceutical composition comprising said one or more ligands.

The invention also relates to a ligand identified by the method described hereinabove.

Yet another aspect of the invention relates to a pharmaceutical composition comprising a ligand identified by the method described hereinabove.

Another aspect of the invention relates to the use of a ligand identified by the method described hereinabove in the preparation of a pharmaceutical composition for use in the treatment of proliferative disorders.

The above methods may be used to screen for a ligand useful as an inhibitor of one or more CDK enzymes, FLT-3, an aurora kinase, GSK-3, PLK or a tyrosine kinase enzyme.

### Synthesis

The compounds of the invention may be prepared by the synthetic route set forth in WO 02/079193 (Cyclacel Limited).

By way of example, compounds of formula I and II can be synthesised, for example, by an adaptation of the Traube synthesis (A. R. Katritzlcy, I. Taher, Can. J. Chem. 1986, 64, 2087 and references cited therein), i.e. by condensation between 1,3-dicarbonyl compounds 1, 1a or acrylates 2, 2a or 3, 3a, and amidine 4, 4a, as shown below in Schemes 1a and 1b.

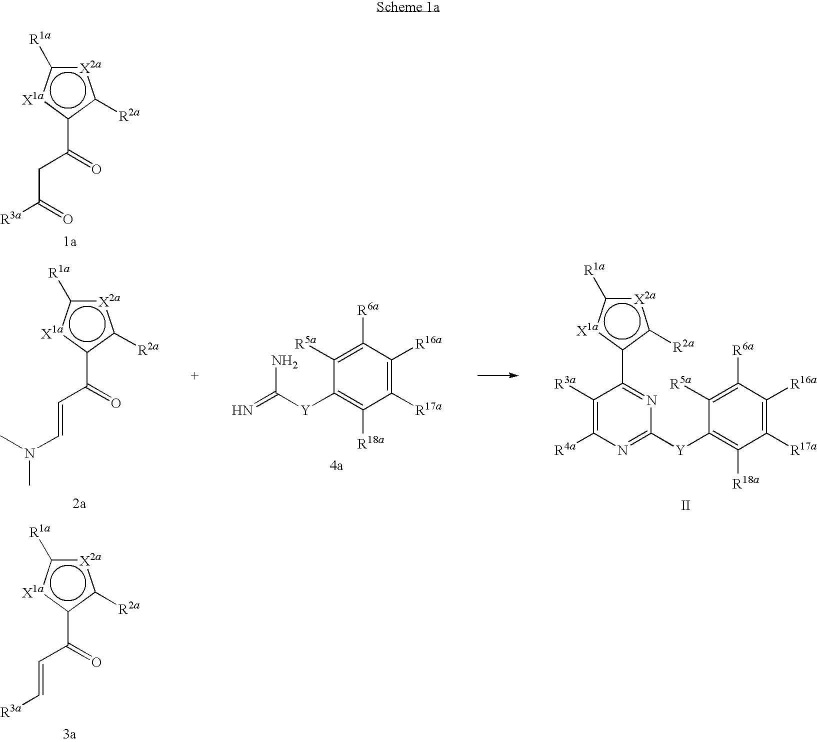

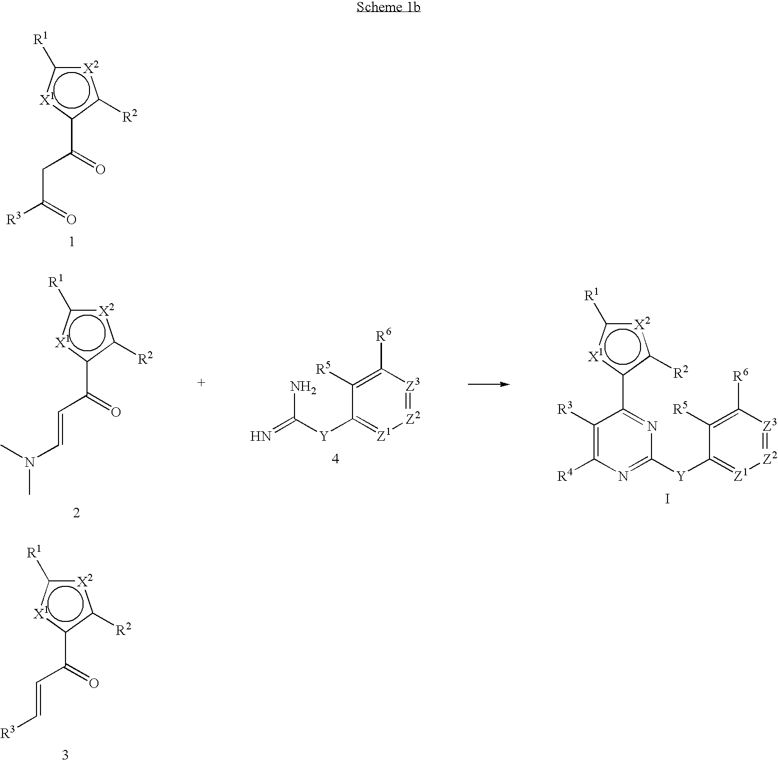

The dicarbonyl compounds 1, 1a in turn can be prepared by many methods known in the art (J. March, In: Advanced Organic Chemistry: Reactions, Mechanism, and Structure, 4^{th} Ed., John Wiley & Sons, Inc., New York, 1992, p. 1283). Acrylates 2, 2a and 3, 3a which are particularly suitable for the purposes of this invention, are obtained from heterocyclic methyl ketones 5 by condensation with tert-butoxybis(dimethylamino)methane 6 (Scheme 2).

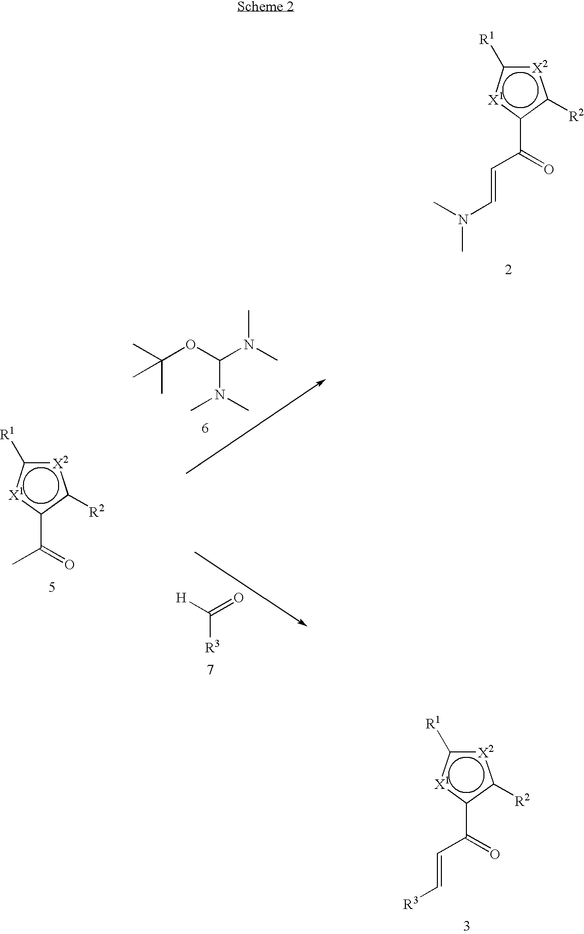

The diamino compounds 4, 4a will be amidines 4c or guanidines 4d, 4e depending on the definition of Y in general structure I or II. Amidines (HN═CRNH₂) can be obtained from readily available amine precursors by condensation with e.g. ketenimines, or by addition of ammonia to suitable nitrites or imidates. Guanidines 4d, 4e (Scheme 3) can be elaborated by a number of methods known in the art. For the purposes of this invention, the most useful route is amination of cyanamide 8 with anilines 9, 9a.

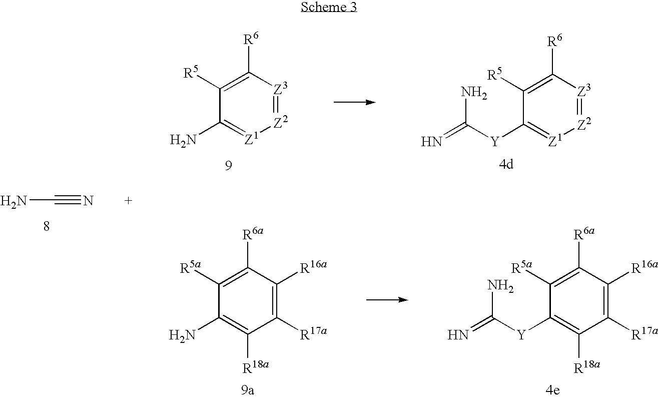

For pyrrole 5, two systems can apply (refer Scheme 4), i.e. the acetyl group which is used to generate the pyrimidine precursors 2, 2a and 3, 3a is either in the pyrrole 3-position (5: X¹═CR⁸, X²═NH; i.e. structure 5b) or in the pyrrole 2-position (X¹═NH, X²═CR⁸; i.e. structure 5c).

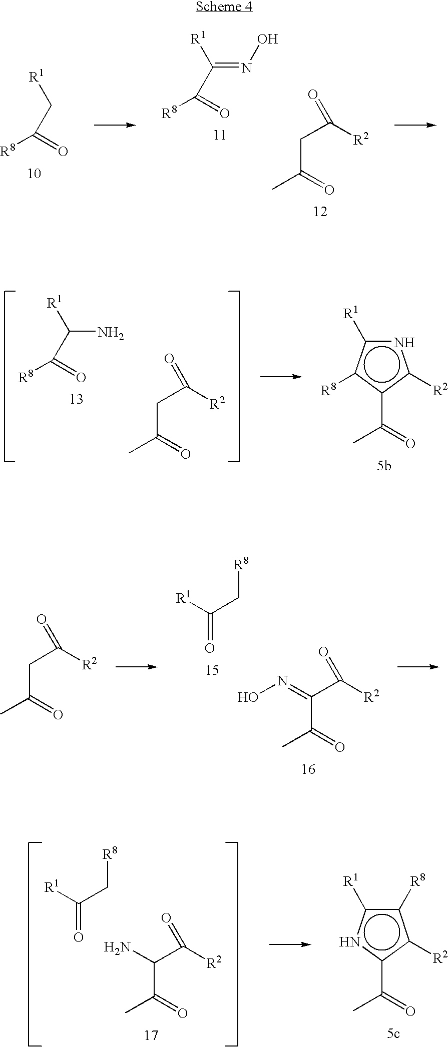

In both cases the pyrrole rings can be assembled using methods known in the art. Particularly relevant is a modification of the Knorr synthesis (refer, e.g. J. A. Joule, G. F. Smith, Heterocyclic Chemistry, 2^{nd} Ed., Van Nostrand Reinhold (UK) Co. Ltd., 1978, pp. 213-215). For the pyrrol-3-yl system, activated (i.e. R¹=COOEt, CN, etc.) carbonyl compounds 10 are first nitrosylated. The resulting oximes 11 are condensed with dicarbonyl compounds 12 in the presence of e.g. zinc-acetic acid or aqueous dithionate, with formation of the reactive α-aminocarbonyl intermediate 13. The R¹ substituent (e.g. COOEt, CN) in the resulting 3-acetylpyrroles 5b can be further manipulated, either directly, or in the context of intermediates 2 or 3, or in the pyrrolopyrimidine products I, II. Thus decarboxylation (R¹=COOEt) will give products with R¹=H, oxidation (R¹=CN) will afford products with R¹=CONH₂, etc. Furthermore, products with R¹=H can be transformed into various derivatives, particularly through electrophilic substitution. Thus derivatives where R¹ is, for example, a halogen, nitro, amino, alkyl, alkylamino, etc., group can be obtained readily. In the case of the pyrrol-2-yl system an analogous situation arises, here an activating group needs to be present in the carbonyl component 15 (e.g. R⁸=COOEt, CN, etc.). This is condensed with oximes 16 (derived from dicarbonyl compounds 14), again with formation of the intermediate 17. The R⁸ substituent in products 5c or derivatives can be manipulated in the same way as the R¹ group in the pyrrol-3-yl system discussed above.

Alternatively, compounds of general structure I, II can be obtained from suitable pyrimidine precursors directly, e.g. from 2,4-disubstituted (halogen, amine, etc.) pyrimidines by successive substitution reactions.

The present invention is further described by way of example.

### EXAMPLES

### General

NMR spectra were recorded using a Varian INOVA-500 instrument. Chemical shifts are reported in parts per million relative to internal tetramethylsilane standard. Mass spectra were obtained using a Waters ZQ2000 single quadrupole mass spectrometer with electrospray ionization (ESI). Analytical and preparative RP-HPLC was performed using Vydac 218TP54 (250×4.6 mm) and 218TP1022 (250×22 mm) columns, respectively. Linear gradient elution using H₂O/MeCN systems (containing 0.1% CF₃COOH) at flow rates of 1 mL/min (analytical) and 9 mL/min (preparative) was performed. Purity was assessed by integration of chromatograms (λ=254 nm). Silica gel (EM Kieselgel 60, 0.040-0.063 mm, Merck) or ISOLUTE pre-packed columns (Jones Chromatography Ltd. UK) were used for flash chromatography.

The compounds of the invention were prepared by a modification of the synthetic approach described in WO 02/0790193 (Cyclacel Limited)

Exemplified compounds are shown in Table I. Characterisation data (NMR, MS, HPLC and MP) are shown in Table II.

### Kinase Assays

The compounds of the invention above were investigated for their ability to inhibit the enzymatic activity of various protein kinases. This was achieved by measurement of incorporation of radioactive phosphate from ATP into appropriate polypeptide substrates. Recombinant protein kinases and kinase complexes were produced or obtained commercially. Assays were performed using 96-well plates and appropriate assay buffers (typically 25 mM β-glycerophosphate, 20 mM MOPS, 5 mM EGTA, 1 mM DTT, 1 mM Na₃VO₃, pH 7.4), into which were added 2-4 μg of active enzyme with appropriate substrates. The reactions were initiated by addition of Mg/ATP mix (15 mM MgCl₂+100 μM ATP with 30-50 kBq per well of [γ-³²P]-ATP) and mixtures incubated as required at 30° C. Reactions were stopped on ice, followed by filtration through p81 filterplates or GF/C filterplates (Whatman Polyfiltronics, Kent, UK). After washing 3 times with 75 mM aq orthophosphoric acid, plates were dried, scintillant added and incorporated radioactivity measured in a scintillation counter (TopCount, Packard Instruments, Pangbourne, Berks, UK). Compounds for kinase assay were made up as 10 mM stocks in DMSO and diluted into 10% DMSO in assay buffer. Data was analysed using curve-fitting software (GraphPad Prism version 3.00 for Windows, GraphPad Software, San Diego Calif. USA) to determine IC₅₀ values (concentration of test compound which inhibits kinase activity by 50%).

### CDK 7 and 9 Assays

CTD peptide substrate (biotinyl-Ahx-(Tyr-Ser-Pro-Thr-Ser-Pro-Ser)₄-NH₂; 1-2 mg/mL) and recombinant human CDK7/cyclin H, CDK9/cyclin T1, or CDK9/cyclin K (0.5-2 μg) were incubated for 45 min at 30° C. in the presence of varying amounts of test compound in 20 mM MOPS pH 7.2, 25 mM β-glycerophosphate, 5 mM EGTA, 1 mM DTT, 1 mM sodium vanadate, 15 mM MgCl₂, and 100 μM ATP (containing a trace amount of ³²PγATP) in a total volume of 25 μL in a 96-well microtiter plate. The reaction was stopped by placing the plate on ice for 2 min. Avidin (50 μg) was added to 6 each well, and the plate was incubated at room temp for 30 min. The samples were transferred to a 96-well P81 filter plate, and washed (4×200 μL per well) with 75 mM phosphoric acid. Microscint 40 scintillation liquid (50 μL) was added to each well, and the amount of ³²P incorporation for each sample was measured using a Packard Topcount microplate scintillation counter.

### GSK-30 Kinase Assay

GSK-3 was obtained from New England Biolabs (UK) Ltd., Hitchin, Herts. The recombinant enzyme was isolated from a strain of E. coli that carries a clone expressing GSK-3β derived from a rabbit skeletal muscle cDNA library [Wang, Q. M.; Fiol, C. J.; DePaoli-Roach, A. A.; Roach, P. J. J. Biol. Chem., 1994, 269, 14566]. Inhibition of GSK-3 function was assessed by measurement of phosphorylation of CREB phosphopeptide KRREILSRRPphosphoSYR in the presence of test compounds. Using a 96-well assay format, GSK3 (7.5 U) was incubated for 30 min at 30° C. in a total volume of 25 μL in 20 mM MOPS pH 7.2, 25 mM β-glycerophosphate, 5 mM EGTA, 1 mM DTT, 1 mM Na₃VO₃, 40 μM CREB peptide, 15 mM MgCl₂ and 100 μM ATP (containing 0.25 μCi [γ-³²P]-ATP) in the presence of varying concentrations of test compound. The samples were transferred to 96-well p81 filter plates (Whatman Polyfiltronics, Kent, UK), and the plates were washed 4 times with 200 μL/well of 75 mM aq orthophosphoric acid. Scintillation liquid (50 μL) was added to each well, and incorporated radioactivity for each sample was determined using a scintillation counter (TopCount, Packard Instruments, Pangboume, Berks, UK).

### Aurora-A (Human) Kinase Assay

This was achieved by measurement of incorporation of radioactive phosphate from ATP into Kemptide substrate (LRRASLG), upon phosphorylation by commercially obtained aurora-A (human, Upstate, Dundee, UK). Assays were performed using 96-well plates and appropriate assay buffers (20 mM Tris, 25 mM β-glycerophosphate, 5 mM EGTA, 1 mM DTT, 1 mM sodium vanadate, pH 7.5), into which were added 2-5 ng of active enzyme with 500 μM substrate (Kemptide). The reactions were initiated by addition of MgATP mix (15 mM MgCl₂+100 μM ATP with 15-25 kBq per well of [γ-³²P]-ATP) and mixtures incubated for 30 min at 30° C. Reactions were stopped by addition of an equal volume of 75 mM aq orthophosphoric acid, followed by filtration through p81 filterplates (Whatman Polyfiltronics, Kent, UK). After washing 4 times with 75 mM aq orthophosphoric acid, plates were dried, scintillant added and incorporated radioactivity measured in a scintillation counter (TopCount, Packard Instruments, Pangbourne, Berks, UK). Compounds for kinase assay were made up as 10 mM stocks in DMSO and diluted into 10% DMSO in assay buffer. Data was analysed using curve-fitting software (XLfit version 4.0.2, IDBS, Guildford, Surrey, UK) to determine IC₅₀ values (concentration of test compound which inhibits kinase activity by 50%).

### Aurora-B (Human) Kinase Assay

This was achieved by measurement of incorporation of radioactive phosphate from ATP into Kemptide substrate (LRRASLG), upon phosphorylation by commercially obtained aurora-B (human, Upstate, Dundee, UK). Assays were performed using 96-well plates and appropriate assay buffers (20 mM Tris, 25 mM β-glycerophosphate, 5 mM EGTA, 1 mM DTT, 1 mM sodium vanadate, pH 7.5), into which were added 75 ng of pre-activated enzyme with 500 μM substrate (Kemptide). The reactions were initiated by addition of MgATP mix (15 mM MgCl₂+100 μM ATP with 15-25 kBq per well of [γ-³²P]-ATP) and mixtures incubated for 60 min at 30° C. Reactions were stopped by addition of an equal volume of 75 mM aq orthophosphoric acid, followed by filtration through p81 filterplates (Whatman Polyfiltronics, Kent, UK). After washing 4 times with 75 mM aq orthophosphoric acid, plates were dried, scintillant added and incorporated radioactivity measured in a scintillation counter (TopCount, Packard Instruments, Pangbourne, Berks, UK). Compounds for kinase assay were made up as 10 mM stocks in DMSO and diluted into 10% DMSO in assay buffer. Data was analysed using curve-fitting software (XLfit version 4.0.2, IDBS, Guildford, Surrey, UK) to determine IC₅₀ values (concentration of test compound which inhibits kinase activity by 50%).

### Pre-Activation of Aurora-B (Human)

Aurora-B (human, Upstate, Dundee, UK) was pre-activated immediately prior to kinase assay in appropriate buffers (20 mM Tris, 25 mM β-glycerophosphate, 5 mM EGTA, 1 mM DTT, 1 mM sodium vanadate, pH 7.5) by incubating 15 μg of enzyme with 4 μg INCENP (Upstate, Dundee, UK) in the presence of MgATP mix (15 mM MgCl₂+100M ATP) for 15 min at 30° C.

### Flt3 Kinase Assay

This was achieved by measurement of incorporation of radioactive phosphate from ATP into myelin basic protein (MBP) substrate, upon phosphorylation by commercially obtained Flt-3 (Upstate, Dundee, UK). Assays were performed using 96-well plates and appropriate assay buffers (20 mM Tris, 25 mM β-glycerophosphate, 5 mM EGTA, 1 mM DTT, 1 mM sodium vanadate, pH 7.5), into which were added 5 ng of active enzyme with 0.4 mg/ml substrate (MBP). The reactions were initiated by addition of MgATP mix (15 mM MgCl₂+100 μM ATP with 15-25 kBq per well of [γ-³²P]-ATP) and mixtures incubated for 30 min at 30° C. Reactions were stopped by addition of an equal volume of 75 mM aq orthophosphoric acid, followed by filtration through p81 filterplates (Whatman Polyfiltronics, Kent, UK). After washing 4 times with 75 mM aq orthophosphoric acid, plates were dried, scintillant added and incorporated radioactivity measured in a scintillation counter (TopCount, Packard Instruments, Pangbourne, Berks, UK). Compounds for kinase assay were made up as 10 mM stocks in DMSO and diluted into 10% DMSO in assay buffer. Data was analysed using curve-fitting software (XLfit version 4.0.2, IDBS, Guildford, Surrey, UK) to determine IC₅₀ values (concentration of test compound which inhibits kinase activity by 50%).

In vitro kinase data for selected compounds of the invention are shown in Table III (Ki values in μM).

Advantageously, selected compounds of the present invention exhibit improved pharmacokinetic properties compared to compounds known in the art. For example, the compounds may exhibit improved bioavailability, solubility and/or half life.

### MTT Cytotoxicity Assay

The compounds of the invention were subjected to a standard cellular proliferation assay using human tumour cell lines obtained from the ATCC (American Type Culture Collection, 10801 University Boulevard, Manessas, Va. 20110-2209, USA). Standard 72-h MT (thiazolyl blue; 3-[4,5-dimethylthiazol-2-yl]-2,5-diphenyltetrazolium bromide) assays were performed (Haselsberger, K.; Peterson, D. C.; Thomas, D. G.; Darling, J. L. Anti Cancer Drugs 1996, 7, 331-8; Loveland, B. E.; Johns, T. G.; Mackay, I. R.; Vaillant, F.; Wang, Z. X.; Hertzog, P. J. Biochemistry International 1992, 27, 501-10). In short: cells were seeded into 96-well plates according to doubling time and incubated overnight at 37° C. Test compounds were made up in DMSO and a ⅓ dilution series prepared in 100 μL cell media, added to cells (in triplicates) and incubated for 72 ho at 37° C. MTT was made up as a stock of 5 mg/mL in cell media and filter-sterilised. Media was removed from cells followed by a wash with 200 μL PBS. MT solution was then added at 20 μL per well and incubated in the dark at 37° C. for 4 h. MTT solution was removed and cells again washed with 200 μL PBS. MT dye was solubilised with 200 μL per well of DMSO with agitation. Absorbance was read at 540 nm and data analysed using curve-fitting software (GraphPad Prism version 3.00 for Windows, GraphPad Software, San Diego Calif. USA) to determine IC₅₀ values (concentration of test compound which inhibits cell growth by 50%).

### Differentiation of Adipocytes and Myotubes

3T3-L1 mouse pre-adipocytes were grown in DMEM supplemented with 10% FCS and pen/strep until fully confluent. Cell differentiation was initiated by the addition of 0.5 mM IBMX (2-isobutyl-1-methylxanthine), 0.25 μM Dexamethasone and 1 μg/ml insulin into the growth media. The differentiation media was replaced after 4 days and 7 days after the initiation of differentiation the cells were grown for additional 3 days in DMEM, 10% FCS and pen/strep.

Rat myotubes were differentiated from L6.G8.C5 myoblasts, which were grown in DMEM, 10% FCS and pen/strep until confluent. The media was then removed, cells washed with PBS and differentiation media containing minimal essential media eagles (alpha modified) supplemented with 2% FCS and pen/strep. The cells were cultured for 3-4 days until >90% of cells have formed multinucleated myotubes. The differentiated cells were then used for determination of glycogen synthase activation after treatment with GSK3 inhibitors.

### Glycogen Synthase Activation in Cultured Cells

HEK293 cells, mouse adipocytes or rat myotubes were treated in 10 cm Petri dishes with different concentrations of GSK3 inhibitors for 90 minutes. At the end of the treatment period the cells were washed and scraped in ice cold PBS buffer supplemented with 20 mM NaF. The cells were pelleted by centrifugation and lysed in 300 μl buffer-50 mM HEPES pH 7.5, 10 mM EDTA, 100 mM NaF, 5 mM DTT, protease inhibitor cocktail (Sigma). After incubation for 30 min on ice the samples were cleared by centrifugation. The activity of glycogen synthase was determined in the soluble fraction at two different concentrations of glucose-6-phosphatase-low (0.1 mM) and high (10 mM). The reaction was carried out for 30 min in a buffer—50 mM Tris pH 7.8, 20 mM EDTA, 25 mM NaF, S mM DTT. The reaction mixture (total volume of 90 μl) consisted as well of 1% glycogen, 0.3 mM UDP-glucose and 0.06 μCi ¹⁴C-UDP-glucose. Reaction was stopped by transfer of 70 μl to a GFC 96-well filter plate, containing 140 μl 100% ethanol an the glycogen was allowed to precipitate for 1 h at 4° C. The wells were washed 2 times with 200 μl 66% ethanol and than let to dry. Subsequently, 100 μl of scintillation liquid were added, and plates were sealed and counted in a Packard Topcounter. Glycogen synthase activation was calculated as the ratio between the incorporation of labelled ¹⁴C-UDP-glucose in glycogen at low and high concentration of UDP-glucose (fractional velocity).

PEPCK Gene Expression Assay—qPCR

PEPCK gene expression was studied in HEPG2 (hepatocarcinoma) cells, seeded in 6-well plate at 1×10⁷ cells per well. The cells were serum starved for 20 hours before treatment with dexamethasone/cAMP (stimulator of PEPCK gene expression) in the presence or absence of insulin or GSK3 inhibitors. After 3 hours treatment the cells were harvested, lysed and RNA extracted using mini RNeasy spin column (Quiagen). The primer set COD2063/COD2064 (350 bp) was used for PEPCK gene. The one step RT-PCR was carried out using the Lightcycler-RNA Master SYBR Green 1 Kit. The qPCR analysis calculates the number of the PCR cycles required for the PCR product amplification to reach logarithmic phase. QPCR for a housekeeping gene—28S—was used for normalisation.

### Results

### Stimulation of Glycogen Synthase Activity in Cellular Systems

The ability of pyrrolo pyrimidines with GSK3 inhibitory activity to stimulate glycogen synthase was determined in HEK293 cells, mouse adipocytes and rat myotubes. The EC₅₀ values (the concentration required to induce half-maximal activation of glycogen synthase) determined are presented in Table IV.

### Effect of Pyrrolo-Pyrimidine GSK3 Inhibitors on PEPCK Gene Expression

PEPCK is a key enzyme in gluconeogenesis in the liver and it is known be negatively regulated by insulin via inhibition of GSK3. The effect of GSK3 inhibitors on PEPCK gene expression was studied in HEPG2 (hepatocarcinoma) cells treated with dexametasone/cAMP (a positive regulator of PEPCK gene expression) in the presence or absence of insulin or GSK3 inhibitors. The level of PEPCK gene transcription expressed as a percentage of the dexametasone-induced stimulation is shown in Table V.

Pyrrolo-pyrimidine inhibitors of GSK3 are efficient in the abolishment of dexametasone/cAMP induced stimulation of PEPCK gene expression in HEPG2 cells as the effect is comparable or even better then the one of the insulin. These results suggest the potential use of GSK3 inhibitors in the regulation of hepatic gluconeogenesis, which is defective and contributes to the hyperglycaemia in diabetic patients.

Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the relevant fields are intended to be within the scope of the following claims.

TABLE 1
Selected Compounds of the Invention

| | |
|---|---|
| 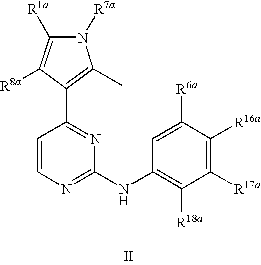 | 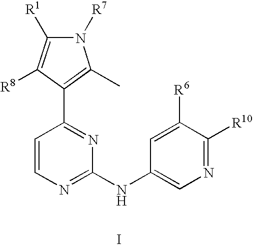 |

| | | | | | | | | | DM Aur |
|---|---|---|---|---|---|---|---|---|---|
| Cpd | | | | | | | | | A IC50 |
| Structure | | R¹/R^{1a} | R⁷/R^{7a} | R⁸/R^{8a} | R⁶/R^{6a} | R¹⁰/R^{16a} | R^{17a} | R^{11a} | (μM) |
| 1 | II | H | H | Me | H | CN | H | H | 10.000 |
| 2 | II | H | H | Me | H | H | CN | H | 10.000 |
| 3 | II | H | H | Me | H | H | CH₂OH | H | |
| 4 | II | H | H | Me | H | Morph | H | H | 0.072 |
| 5 | II | CO₂Et | H | Ph | H | H | NO₂ | H | |
| 6 | II | CO₂Et | H | Ph | H | OH | H | H | 1.209 |
| 7 | II | CO₂Et | H | Ph | H | H | OH | H | |
| 8 | II | CO₂Et | H | Ph | H | H | F | H | 10.000 |
| 9 | II | CO₂Et | H | Ph | H | I | H | H | 10.000 |
| 10 | II | CO₂Et | H | Ph | H | CF₃ | H | H | 10.000 |
| 11 | II | CO₂Et | H | Ph | H | OH | NO₂ | H | 0.712 |
| 12 | II | CO₂Et | H | Ph | H | NMe₂ | H | H | 0.777 |
| 13 | II | H | H | Ph | H | H | NO₂ | H | 0.859 |
| 14 | II | H | H | Ph | H | I | H | H | |
| 15 | II | H | H | Ph | H | OH | H | H | 1.542 |
| 16 | II | H | H | Ph | H | F | H | H | |
| 17 | II | CH₂NMe₂ | H | Me | H | H | NO₂ | H | 0.102 |
| 18 | II | CN | H | Me | H | NMe₂ | NO₂ | H | |
| 19 | II | NO₂ | H | Me | H | NMe₂ | NO₂ | H | |
| 20 | II | CN | Me | Me | H | NMe₂ | H | H | |
| 22 | II | CO₂Et | H | Ph | H | F | H | H | |
| 24 | II | CN | H | Me | H | Morph | H | H | |
| 26 | I | H | H | Me | H | OMe | — | — | |
| 27 | II | CN | H | Me | NO₂ | H | H | Me | 10.000 |
| 28 | I | CN | H | Me | H | OMe | — | — | 0.453 |
| 29 | II | CN | H | Me | OMe | H | H | Me | 6.466 |
| 30 | I | CN | H | Me | H | Cl | — | — | 0.861 |
| 31 | II | CN | H | Me | H | NHMe | CF₃ | H | 0.125 |
| 32 | II | CN | H | Me | H | H | 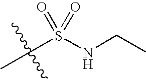 | H | 0.413 |
| 33 | I | CN | H | Me | Me | Cl | — | — | 1.705 |
| 34 | II | CN | H | Me | H | H | 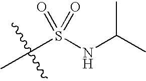 | H | 3.323 |
| 36 | II | CN | H | Me | H | 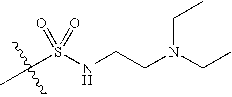 | H | H | 0.786 |
| 37 | II | CO₂(CH₂)₂OMe | H | Ph | H | Morph | H | H | 0.853 |
| 38 | I | CN | H | Me | H | N—Ac | — | — | 0.177 |
| | | | | | | Piperizine | | | |
| 39 | I | CN | Me | Me | H | N—Ac | — | — | 0.234 |
| | | | | | | Piperizine | | | |
| 40 | II | CN | H | Me | H | N-Bz | H | H | 0.276 |
| | | | | | | Piperizine | | | |
| 41 | II | CN | H | Me | H | N—Ac | H | H | 0.048 |
| | | | | | | Piperizine | | | |
| 42 | II | H | H | Ph | H | Morph | H | H | 1.87 |
| 43 | II | CO₂Et | H | Me | H | Morph | H | H | 0.265 |
| 44 | II | H | H | Me | H | Morph | Cl | H | 0.261* |
| 45 | II | H | H | Me | Cl | Morph | Cl | H | >10* |
| 46 | II | CO₂Et | H | Me | H | Morph | Cl | H | 3.030* |
| 47 | II | CO₂Et | H | Me | Cl | Morph | Cl | H | >10* |
| 48 | II | H | Me | Me | H | Morph | H | H | 0.222* |
| 49 | II | H | Me | Me | H | Morph | Cl | H | 0.531* |
| 50 | II | H | H | Me | H | Morph | H | Me | >10* |
| 51 | II | H | H | Me | H | bis-methyl | H | H | 0.230* |
| | | | | | | Morph | | | |
| 52 | II | CN | H | Me | OMe | OMe | OMe | H | 0.168* |
| 53 | II | CN | Me | Me | OMe | OMe | OMe | H | 0.125* |
| 54 | II | CO₂Et | H | Me | H | Morph | OMe | H | 0.091* |
| 55 | II | CO₂Et | H | Me | OMe | H | OMe | H | |
| 56 | II | H | H | Me | OMe | OMe | OMe | H | |
| 57 | II | H | Me | Me | OMe | OMe | OMe | H | |
| 58 | II | CO₂Et | H | Me | OMe | OMe | OMe | H | |
| 59 | II | H | H | Me | H | Morph | OMe | H | |
| 60 | II | H | H | Me | H | N—Ac— | H | H | |
| | | | | | | piperazine | | | |

*Human aurora kinase A

TABLE II
Characterisation Data for Selected Compounds of the Invention

| | | | | HPLC | MP |
|---|---|---|---|---|---|
| No. | Solvent | NMR (δ) | MS | (min) | (° C.) |
| 4 | d6- | 2.17 (3H, s, CH₃), 2.38 (3H, s, CH₃), 3.02 (4H, m, | m/z | 14.35 | |
| | DMSO | CH₂NCH₂), 3.72 (4H, m, CH₂OCH₂), 6.44 (1H, s, | (MALDI) | | |
| | | Pyrrole CH), 6.69 (1H, d, Pyrim CH, J = 5.4), 6.86 (2H, | 350.06 | | |
| | | d, ArH, J = 7.4), 7.60 (2H, d, ArH, J = 7.4), 8.24 (1H, d, | (M + H) | | |
| | | Pyrim CH, J = 5.4), 8.97 (1H, s, NH), 10.67 (1H, s, NH) | C₂₀H₂₃N₅O = | | |
| | | | 349.43 | | |
| 5 | CDCl₃ | 1.06 (3H, t, CH₃, J = 7.1), 2.57 (3H, s, CH₃), 4.11 (2H, q, | m/z | — | 248-250 |
| | | CH₂, J = 7.3), 6.14 (1H, d, Pyrimidine, J = 5.4), 7.19-7.27 | (MALDI) | | |
| | | (5H, m, ArH), 7.38 (1H, t, ArH, J = 8.1), 7.62 (1H, d, | 444.09 | | |
| | | ArH, J = 8.1), 7.78 (1H, d, ArH, J = 8.1), 8.01 (1H, bs, | C₂₄H₂₁N₅O₄₌ | | |
| | | pyrrole-NH), 8.70 (1H, d, Pyrimidine-H, J = 5.4), 9.04 | 443.45 | | |
| | | (1H, s, NH) | | | |
| 6 | CD₃OD | 1.03 (3H, t, CH₃, J = 7.1), 2.45 (3H, s, CH₃), 4.04 (2H, q, | m/z | — | 245-248 |
| | | CH₂, J = 7.1), 5.97 (1H, d, Pyrimidine, J = 4.9), 6.62 (2H, | (MALDI) | | |
| | | d, ArH, J = 7.3), 7.17, (2H, d, ArH, J = 7.2), 7.31 (5H, m, | 413.86 | | |
| | | ArH), 8.00 (1H, d, Pyrimidine, J = 5.1), 8.94 (1H, s), 8.97 | C₂₄H₂₂N₄O₃₌ | | |
| | | (1H, s), 11.99 (1H, s, pyrrole NH) | 414.46 | | |
| 7 | CDCl₃ | 1.25 (3H, t, CH₃, J = 7.1), 2.58 (3H, s, CH₃), 4.17 (2H, q, | m/z | — | 123.1-124.2 |
| | | CH₂, J = 7.1), 6.17 (1H, d, Pyrimidine, J = 5.1), 6.48 (2H, | (MALDI) | | |
| | | dd, ArH, J = 8.1, 1.2), 6.89 (2H, dd, ArH, J = 8.1, 1.2), | 415.56 | | |
| | | 7.09 (1H, bs), 7.26 (1H, t, ArH, J = 8.1), 7.27 (1H, m), | C₂₄H₂₂N₄O₃₌ | | |
| | | 7.29-7.32 (5H, m, ArH), 8.06 (1H, d, Pyrimidine, | 414.46 | | |
| | | J = 5.1), 9.12 (1H, s, NH) | | | |
| 8 | CDCl₃ | 1.13 (3H, t, CH₃, J = 7.1), 2.61 (3H, s, CH₃), 4.17 (2H, q, | m/z | — | 200-203 |
| | | CH₂ J = 7.1), 6.14 (1H, d, Pyrimidine, J = 5.4), 6.70 (1H, | (MALDI) | | |
| | | m, ArH), 7.10 (1H, m, ArH), 7.18-7.31 (7H, m, ArH, | 416.49 | | |
| | | pyrrole-NH), 7.69 (1H, m, ArH), 7.69 (1H, m, ArH), | C₂₄H₂₁FN₄O₂₌ | | |
| | | 8.07 (1H, d, Pyrimidine, J = 5.4), 9.17 (1H, s, NH) | 416.45 | | |
| 9 | CDCl₃ | 1.14 (3H, t, CH₃, J = 7.1), 2.57 (3H, s, CH₃), 4.17 (2H, q, | m/z | — | 195.5-198.4 |
| | | CH₂, J = 7.1), 6.18 (1H, d, Pyrimidine, J = 5.1), 7.13 (1H, | (MALDI) | | |
| | | s, pyrrole NH), 7.25-7.35 (7H, m, ArH), 7.56 (2H, d, | 524.35 | | |
| | | ArH, J = 8.8), 8.07 (1H, d, Pyrimidine, J = 5.4), 9.19 (1H, | C₂₄H₂₁IN₄O₂₌ | | |
| | | s, NH) | 523.2 | | |
| 10 | CDCl₃ | 1.14 (3H, t, CH₃, J = 7.1), 2.59 (3H, s, CH₃), 4.19 (2H, q, | m/z | — | 227-230 |
| | | CH₂, J = 7.1), 6.23 (1H, d, Pyrimidine, J = 5.1), 7.25-7.35 | (MALDI) | | |
| | | (6H, m, ArH, pyrrole NH), 7.52 (2H, d, ArH, J = 8.8), | 465.94 | | |
| | | 7.63 (2H, d, ArH, J = 8.8), 8.11 (1H, d, Pyrimidine, | C₂₅H₂₁F₃N₄O₂₌ | | |
| | | J = 5.4), 9.21 (1H, s, NH) | 466.45 | | |
| 11 | CDCl₃ | 1.14 (3H, t, CH₃, J = 7.0), 2.60 (3H, s, CH₃), 4.17 (2H, q, | m/z | — | 202-205 |
| | | CH_{2,} J = 7.0), 6.18 (1H, d, Pyrimidine, J = 5.4), 7.09 (2H, | (MALDI) | | |
| | | d, ArH, J = 9.0), 7.14 (1H, bs), 7.24-7.32 (2H, m, ArH), | 458.77 | | |
| | | 7.58 (1H, dd, ArH, J = 9.0, 2.8), 8.06 (1H, d, ArH, | C₂₄H₂₁N₅O₅₌ | | |
| | | J = 2.9), 8.56 (1H, d, Pyrimidine, J = 5.1), 9.15 (1H, bs), | 459.45 | | |
| | | 10.41 (1H, bs, NH) | | | |
| 12 | CDCl₃ | 2.56 (3H, s, CH₃), 2.91 (6H, s, CH₃), 3.27 (3H, s, CH₃), | m/z | — | 212-213 |
| | | 3.46 (2H, t, CH₂, J = 4.6), 4.26 (2H, t, CH₂, J = 4.6), 6.01 | (MALDI) | | |
| | | (1H, d, Pyrimidine, J = 5.1), 6.75 (2H, d, ArH, J = 8.5), | 469.86 | | |
| | | 7.26-7.36 (8H, m, ArH, pyrrole NH), 7.98 (1H, d, | C₂₇H₂₉N₅O₃₌ | | |
| | | Pyrimidine, J = 5.1), 9.14 (1H, s, NH) | 471.55 | | |
| 13 | CDCl₃ | 2.50 (3H, s, CH₃), 6.45 (1H, d, Pyrimidine, J = 5.4), 6.75 | m/z (ES) | — | 201-203 |
| | | (1H, d, Pyrrole H, J = 2.5), 6.92 (2H, m, ArH), 7.06 (1H, | 372.68 | | |
| | | bs, pyrrole NH), 7.26-7.39 (7H, m, ArH), 8.45 (1H, bs, | C₂₁H₁₇N₅O₂₌ | | |
| | | NH), 8.16 (1H, d, Pyrimidine, J = 5.1) | 371.39 | | |
| 14 | CDCl₃ | 2.52 (3H, s, CH₃), 6.46 (1H, d, Pyrimidine, J = 5.4), 6.76 | m/z (ES) | — | 108.2-109.5 |
| | | (1H, d, Pyrrole H, J = 2.4), 7.10 (1H, bs, Pyrrole NH), | 453.19 | | |
| | | 7.26-7.30 (1H, m, ArH), 7.50 (2H, m, ArH), 8.15 (1H, | C₂₁H₁₇IN₄₌ | | |
| | | bs, NH), 8.17 (1H, d, Pyrimidine, J = 5.2) | 452.29 | | |
| 15 | d6- | 2.36 (3H, s, CH₃), 6.27 (1H, d, Pyrimidine, J = 5.4), 6.54 | m/z (ES) | — | 194.1-195.6 |
| | DMSO | (2H, d, ArH, J = 8.8), 6.77 (1H, d, Pyrrole H), 7.14-7.17 | 343.29 | | |
| | | (3H, m, ArH), 7.24-7.27 (4H, m, ArH), 8.10 (1H, d, | C₂₁H₁₈N₄O₌ | | |
| | | Pyrimidine, J = 5.1), 8.87 (1H, s,), 8.93 (1H, s), 11.09 | 342.39 | | |
| | | (1H, s, NH) | | | |
| 16 | CDCl₃ | 2.59 (3H, s, CH₃), 6.50 (1H, d, Pyrimidine, J = 5.6), 6.76 | m/z (ES) | — | 196.6-198.1 |
| | | (1H, d, Pyrrole H), 7.22-7.31 (5H, m, ArH), 7.38 (1H, t, | 343.29 | | |
| | | ArH, J = 8.1), 7.47 (1H, bs, Pyrrole NH), 7.70 (1H, dd, | C₂₁H₁₇FN₄₌ | | |
| | | ArH, J = 8.3, 2.2), 7.82 (1H, dd, ArH, J = 8.3, 2.2), 8.19 | 344.38 | | |
| | | (2H, d, Pyrimidine, J = 5.4), 8.72 (1H, t, ArH, J = 2.2) | | | |
| 17 | CDCl₃ | 2.22 (3H, s, CH₃), 2.30 (6H, s, CH₃), 2.48 (3H, s, CH₃), | m/z (ES) | — | 88.3-92.6. |
| | | 3.42 (2H, s, CH₂), 6.86 (1H, d, Pyrimidine H, J = 5.4), | 366.50 | | |
| | | 7.28 (1H, bs, NH), 7.45 (1H, t, ArH, J = 8.1), 7.80 (1H, | C₁₉H₂₂N₆O₂ = | | |
| | | m, ArH), 7.84 (1H, m, ArH), 8.38 (1H, d, Pyrimidine H, | 366.42 | | |
| | | J = 5.4), 8.84 (1H, bs, NH) | | | |
| 18 | d6- | 2.42 (3H, s, CH₃), 2.51 (3H, s, CH₃), 2.73 (6H, s, | m/z (ES) | 17.45 | — |
| | DMSO | N(CH₃)₂), 6.85 (1H, d, Pyrimidine, J = 5.4), 7.21 (1H, d, | 376.41 | | |
| | | ArH, J = 8.3), 7.73, (1H, dd, ArH, J = 8.3, 2.4), 8.36 (1H, | (M − H) | | |
| | | d, ArH, J = 2.4), 8.42 (1H, d, Pyrimidine, J = 5.4), 9.59 | C₁₉H₁₉N₇O₂ = | | |
| | | (1H, s, NH), 12.11 (1H, s, Pyrrole NH) | 377.40 | | |
| 19 | d6- | 2.43, (3H, s, CH₃), 2.25, 3H, s, CH₃), 2.75, (6H, s, | m/z (ES) | 17.48 | — |
| | DMSO | (CH₃)₂), 6.90, (1H, d, Pyrimidine H, J = 5.4), 7.25. (1H, | 398.64 | | |
| | | d. ArH, J = 8.8), 7.77 (1H, dd, ArH, J = 8.8, 2.4), 8.42 | (M + H) | | |
| | | (1H, d, ArH, J = 2.4), 8.50 (1H, d, Pyrimidine H, J = 5.4), | C₁₈H₁₉N₇O₄ = | | |
| | | 9.74 (1H, s, NH), 13.06 (1H, s, Pyrrole NH) | 397.39 | | |
| 20 | CDCl₃ | 2.32 (3H, s, CH₃), 2.40 (3H, s, CH₃), 2.93 (6H, s, CH₃), | m/z (ES) | — | — |
| | | 3.58 (3H, s, CH₃), 6.59 (1H, d, Pyrimidine H, J = 5.1), | 347.74 | | |
| | | 6.73 (2H, d, ArH, J = 8.8), 7.36 (2H, d, ArH, J = 8.8), 7.48 | (M + H) | | |
| | | (1H, bs, NH), 8.33 (1H, d, Pyrimidine H, J = 5.1) | C₂₀H₂₂N₆ = | | |
| | | | 346.43 | | |
| 22 | CDCl₃ | 1.14 (3H, t, CH₃, J = 7.1), 2.55 (3H, s, CH₃), 4.18 (2H, q, | m/z (ES) | — | 220-225 |
| | | CH₂, J = 7.1), 6.15 (1H, d, Pyrimidine H, J = 5.1), 6.98 | 416.86 | | |
| | | (2H, t, ArH, J = 8.5) 7.07 (1H, bs, Pyrrole NH), 7.26-7.32 | C₂₄H₂₁FN₄O_{2 =} | | |
| | | (5H, m, ArH), 7.41 (2H, m, ArH), 8.06 (1H, d, | 416.45 | | |
| | | Pyrimidine H, J = 5.4), 9.19 (1H, s, NH) | | | |
| 24 | d6- | 2.38 (3H, s, CH₃), 2.49 (3H, s, CH₃), 3.00 (4H, m, | m/z (ES) | — | — |
| | DMSO | CH₂NCH2), 3.72 (4H, m, CH₂OCH₂), 6.74 (1H, d, | 375.21 | | |
| | | Pyrimidine H, J = 4.9), 6.86 (2H, d, ArH, J = 8.8), 7.56 | (M + H) | | |
| | | (2H, d, ArH, J = 8.8), 8.35 (1H, d, Pyrimidine H, J = 4.9), | C₂₁H₂₂N₆O = | | |
| | | 9.16 (1H, s, NH), 12.07 (1H, bs, Pyrrole NH) | 374.44 | | |
| 26 | d6- | 2.16 (3H, s, CH₃), 2.37 (3H, s, CH₃), 3.80 (3H, s, | m/z (ES) | 11.08* | — |
| | DMSO | OCH₃), 6.45 (1H, s, Pyrrole H), 6.75 (1H, d, Pyrimidine | 296.11 | | |
| | | H, J = 5.5), 6.76 (1H, d, ArH, J = 9.0), 8.04 (1H, dd, Ar, | (M + H) | | |
| | | J = 9.0, 3.0), 8.28 (1H, d, Pyrimdine H, J = 5.5), 8.44 (1H, | C₁₆H₁₇N₅O = | | |
| | | d, ArH, J = 3.0), 9.13 (1H, s, NH), 10.69 (1 H, br s, | 295.34 | | |
| | | Pyrrole NH) | | | |
| 27 | d6- | 2.24 (3H, s, CH₃), 2.33 (3H, s, CH₃), 2.39 (3H, s, CH₃), | m/z (ES) | 18.57 | — |
| | DMSO | 6.92 (1H, d, Pyrimidine H, J = 5.4), 7.51 (1H, d, ArH, | 349.43 | | |
| | | J = 8.8), 7.90 (1H, dd, ArH, J = 8.8, 2.4), 8.43 (1H, d, | (M + H) | | |
| | | Pyrimidine H, J = 5.4), 8.59 (1H, d, ArH, J = 2.4), 8.99 | C₁₈H₁₆N₆O₂ = | | |
| | | (1H, s, NH), 12.23 (1H, bs, Pyrrole NH) | 348.36 | | |
| 28 | d6- | 2.29 (3H, s, CH₃), 2.39 (3H, s, CH₃), 3.81 (3H, s, | m/z (ES) | 15.28 | — |
| | DMSO | OCH₃), 6.79 (1H, d, Pyridine H, J = 8.8), 6.84 (1H, d, | 321.38 | | |
| | | Pyrimidine H, J = 5.4), 8.01 (1H, dd, Pyridine H, J = 8.8, | (M + H) | | |
| | | 2.9), 8.39 (1H, d, Pyrimidine H, J = 5.4), 8.41 (1H, d, | C₁₇H₁₆N₆O = | | |
| | | Pyridine H, J = 2.9), 9.39 (1H, s, NH), 12.22 (1H, s, | 320.35 | | |
| | | Pyrrole NH) | | | |
| 29 | d6- | 2.14 (3H, s, CH₃), 2.23 (3H, s, CH₃), 2.32 (3H, s, CH₃), | m/z (ES) | 17.52 | — |
| | DMSO | 3.70 (3H, s, OCH₃), 6.67 (1H, dd, ArH, J =, 8.3, 2.9), | 334.36 | | |
| | | 6.84 (1H, d, Pyrimidine H, J = 5.4), 7.11 (1H, d, ArH, | (M + H) | | |
| | | J = 2.9), 7.13 (1H, d, ArH, J = 8.3), 8.33 (1H, d, | C₁₉H₁₉N₅O = | | |
| | | Pyrimidine H, J = 5.4), 8.84 (1H, bs, NH), 12.23 (1H, bs, | 333.39 | | |
| | | Pyrrole NH) | | | |
| 30 | d6- | 2.31 (3H, s, CH₃), 2.42 (1H, s, CH₃), 6.94 (1H, d, | m/z (ES) | 18.39 | — |
| | DMSO | Pyrimidine H, J = 4.9), 7.44 (1H, d, Pyridine H, J = 8.8), | 325.35 | | |
| | | 8.26 (1H, dd, Pyridine H, J = 8.8, 2.9), 8.47 (1H, d, | (M + H) | | |
| | | Pyrimidine H, J = 4.9), 8.74 (1H, d, Pyridine H, J = 2.9), | C₁₆H₁₃ClN₆ = | | |
| | | 9.81 1H, s, NH), 12.24 (1H, bs, Pyrrole NH) | 324.77 | | |
| 31 | d6- | 2.28 (3H, s, CH₃), 2.38 (3H, s, CH₃), 2.75 (3H, d, | m/z (ES) | 18.37 | — |
| | DMSO | NCH₃, J = 4.4), 5.28 (1H, d, NH, J = 4.4), 6.71 (1H, d, | 387.46 | | |
| | | ArH, J = 8.3), 6.76 (1H, d, Pyrimidine CH, J = 5.4), 7.68 | (M + H) | | |
| | | (1H, d, ArH, J = 8.3), 7.84 (1H, s, ArH), 8.36 (1H, d, | C₁₉H₁₇F₃N₆ = | | |
| | | Pyrimidine, J = 5.4), 9.21 (1H, s, NH), 12.17 (1H, s, | 386.37 | | |
| | | Pyrrole NH) | | | |
| 32 | d6- | 0.98 (3H, t, CH₃, J = 7.2), 2.33 (3H, s, CH₃), 2.43 (3H, s, | m/z (ES) | 17.50 | — |
| | DMSO | CH₃), 2.79 (2H, q, CH₂, J = 7.2), 6.91 (1H, d, Pyrimidine | 397.45 | | |
| | | H, J = 5.4), 7.37 (1H, d, ArH, J = 8.3), 7.47 (1H, s, | (M + H) | | |
| | | SO₂NH), 7.49 (1H, d, ArH, J = 8.3), 7.94 (1H, d, ArH, | C₁₉H₂₀N₆O₂S = | | |
| | | J = 8.3), 8.35 (1H, s, ArH), 8.46 (1H, d, Pyrimidine H, | 396.47 | | |
| | | J = 5.4), 9.81 (1H, s, NH), 12.20 (1H, bs, Pyrrole NH) | | | |
| 33 | d6- | 6.94 (1H, d, Pyrimidine CH, J = 5.4), 8.20 (1H, d, | m/z (ES) | 18.62 | — |
| | DMSO | Pyridine CH, J = 2.4), 8.48 (1H, d, Pyrimidine CH, | 339.40 | | |
| | | J = 5.4), 8.59 (1H, d, Pyridine CH, J = 2.4), 9.73 (1H, s, | (M + H) | | |
| | | NH), 12.28 (1H, s, Pyrrole NH) | C₁₇H₁₅ClN₆ = | | |
| | | | 338.79 | | |
| 34 | d6- | 0.96 (6H, d, (CH₃)₂, J = 7.1), 2.30 (3H, s, CH₃), 2.45 (3H, | m/z (ES) | 18.35 | — |
| | DMSO | s, CH₃), 3.24 (1H, m, CH), 6.92 (1H, d, Pyrimidine H, | 411.32 | | |
| | | J = 5.4), 7.36 (1H, d, ArH, 8.3), 7.46 (1H, d, ArH, J = 8.3), | (M + H) | | |
| | | 7.48 (1H, dd, ArH, J = 8.3, 8.3), 8.37 (1H, s, ArH), 8.48 | C₂₀H₂₂N₆O₂S = | | |
| | | (1H, d, Pyrimidine H, J = 5.4), 9.80 (1H, s, NH), 12.21 | 410.49 | | |
| | | (1H, bs, Pyrrole NH) | | | |
| 36 | d6- | 0.88 (6H, t, 2 × CH₃, J = 7.1), 2.32 (3H, s, CH₃), 2.44 (3H, | m/z (ES) | 15.52 | — |
| | DMSO | s, CH₃), 2.49 (2H, m, CH₂), 2.78 (4H, t, 2xNCH₂, | 468.27 | | |
| | | J = 7.1), 3.34 (2H, m, CH₂), 6.97 (1H, d, Pyrimidine H, | (M + H) | | |
| | | J = 5.4), 7.70 (2H, d, ArH, J = 8.8), 7.95 (2H, d, ArH, | C₂₃H₂₉N₇O₂S = | | |
| | | J = 8.8), 8.50 (1H, d, Pyrimidine H, J = 5.4, 9.93 (1H, s, | 467.59 | | |
| | | NH), 12.26 (1H, bs, Pyrrole NH) | | | |
| 37 | d6- | 2.46 (3H, s, CH₃), 3.01 (4H, m, CH₂NCH₂), 3.17 (3H, s, | m/z (ES) | 17.08 | — |
| | DMSO | OCH₃), 3.38 (2H, t, CO₂CH₂, J = 4.8), 3.73 (4H, m, CH— | 512.10 | | |
| | | ₂OCH₂), 4.15 (2H, t, CH₂OMe), 5.98 (1H, d, Pyrimidine | (M − H) | | |
| | | H, J = 4.9), 6.82 (2H, d, ArH, J = 8.8), 7.19 (2H, d, ArH, | C₂₉H₃₁N_{%}O₄ = | | |
| | | J = 8.8), 7.28 (3H, m, ArH), 7.41 (2H, m, ArH), 8.03 | 513.59 | | |
| | | (1H, d, Pyrimidine H, J = 4.9), 9.09 (1H, s, NH), 12.04 | | | |
| | | (1H, S, Pyrrole NH) | | | |
| 38 | d6- | 2.04 (3H, s, CH₃), 2.28 (3H, s, CH₃), 2.38 (3 H, s, CH₃), | m/z (ES) | 9.71* | — |
| | DMSO | 3.37 (2 H, m, Piperazine), 3.44 (2 H, m, Piperazine), | 415.08 | | |
| | | 3.53 (4 H, m, Piperazine), 6.77 (1 H, d, J 5.5, | (M − H), | | |
| | | pyrimidine), 6.85 (1 H, d, J 9.0, ArH), 7.90 (1 H, dd, J | 417.12 | | |
| | | 3.0, 9.0, PhP), 8.36 (1 H, d, J 5.5, pyrimdine), 8.38 (1 | (M + H) | | |
| | | H, d, J 3.0, ArH), 9.19 (1 H, s, NH), 12.17 (1 H, br s, | C₂₂H₂₄N₈O = | | |
| | | Pyrrole NH) | 416.48 | | |
| 39 | d6- | 2.04 (3H, s, CH₃), 2.26 (3H, s, CH₃), 2.40 (3H, s, CH₃), | m/z (ES) | 11.09* | — |
| | DMSO | 3.37 (2H, m, Piperazine H), 3.44 (2H, m, Piperazine H), | 431.07 | | |
| | | 3.53 (4H, m, Piperazine H), 3.63 (3H, s, CH₃), 6.75 (1H, | (M + H) | | |
| | | d, Pyrimidine H, J = 5.5), 6.84 (1H, d, ArH, J = 9.0), 7.90 | C₂₃H₂₆N₈O = | | |
| | | (1H, dd, ArH, J = 9.5, 3.0), 8.38 (1H, d, Pyrimdine H, | 430.51 | | |
| | | J = 5.0), 8.40 (1H, d, ArH, J = 3.0), 9.25 (1H, s, NH) | | | |
| 40 | d6- | 2.29 (3H, s, CH₃), 2.39 (3H, s, CH₃), 3.05 (4H, m, | m/z (ES) | 11.83* | — |
| | DMSO | Piperazine H), 3.33 (4H, m, Piperazine H), 3.63 (2H, s, | 462. 15 | | |
| | | CH₂), 6.75 (1H, d, Pyrimidine H, J = 5.0), 6.84 (1 H, d, | (M − H), | | |
| | | ArH, J = 9.5), 7.25 (1H, m, ArH), 7.33 (4 H, m, ArH), | 464.27 | | |
| | | 7.54 (1 H, d, ArH, J = 8.5), 8.35 (1 H, d, Pyrimdine H, | (M + H) | | |
| | | J = 5.5), 9.15 (1H, s, NH), 12.16 (1H, s, Pyrrole NH) | C₂₈H₂₉N₇ = | | |
| | | | 463.58 | | |
| 41 | d6- | 2.03 (3H, s, CH₃), 2.30 (3H, s, CH₃), 2.40 (3H, s, CH₃), | m/z (ES) | 10.87* | — |
| | DMSO | 2.98 (2H, t, Piperazine H, J = 5.0), 3.06 (2H, t, Piperazine | 414.12 | | |
| | | H, J = 5.0), 3.56 (4 H, dd, Piperazine H, J = 5.0, 5.5), 6.76 | (M − H), | | |
| | | (1H, d, Pyrimidine H, J = 5.5), 6.90 (1H, d, ArH, J = 9.5), | 416. 0 | | |
| | | 7.56 (1H, d, ArH, J = 9.0), 8.36 (1H, d, Pyrimdine H, | (M + H) | | |
| | | J = 5.0), 9.20 (1H, s, NH), 12.17 (1H, br s, Pyrrole NH). | C₂₃H₂₅N₇O = | | |
| | | | 415.49 | | |
| 42 | d6- | 2.30 (3H, s, CH₃), 2.99 (4H, m, CH₂NCH₂), 3.72 (4H, | m/z (ES) | 16.74 | — |
| | DMSO | m, CH₂OCH₂), 6.28 (1H, d, Pyrimidine H, J = 5.4), 6.77 | 412.01 | | |
| | | (2H, d, ArH, J = 8.8), 6.79 (1H, d, Pyrrole H, J = 2.4), | (M + 2H) | | |
| | | 7.15-7.26 (5H, m, ArH), 7.43 (2H, d, ArH, J = 8.8), 8.12 | C₂₅H₂₅N₅O = | | |
| | | (1H, d, Pyrimidine H, J = 5.4), 9.05 (1H, s, NH), 11.12 | 411.50 | | |
| | | (1H, bs, Pyrrole NH) | | | |
| 43 | d6- | 1.28 (3H, t, CH₃, J = 7.3), 2.38 (3H, s, CH₃), 2.42 (3H, s, | m/z (ES) | 12.74 | — |
| | DMSO | CH₃), 3.02 (4H, m, CH₂NCH₂), 3.72 (4H, m, | 420.00 | | |
| | | CH₂OCH₂), 4.25 (2H, q, OCH₂, J = 7.3), 6.71 (1H, d, | (M − H) | | |
| | | Pyrimidine H, J = 4.9), 6.88 (2H, d, ArH, J = 8.8), 7.59 | C₂₃H₂₇N₅O_{3 -}= | | |
| | | (2H, d, ArH, J = 8.8), 8.34 (1H, d, Pyrimidine H, J = 4.9), | 421.49 | | |
| | | 9.16 (1H, s, NH), 11.64 (1H, s, Pyrrole NH) | | | |
| 44 | d6- | 2.19 (3H, s, CH₃), 2.41 (3H, s, CH₃), 2.90 (4H, m, | m/z (ES) | 10.26* | — |
| | DMSO | CH₂NCH₂), 3.72 (4H, m, CH₂OCH₂), 6.48 (1H, s, | 383.94 | | |
| | | Pyrrole H), 6.79 (1H, d, Pyrimidine H, J = 5.4), 7.11 (1H, | C₂₀H₂₂ClN₅O = | | |
| | | d, ArH, J = 8.8), 7.55 (1H, dd, ArH, J = 8.8, 2.4), 8.12 | 383.87 | | |
| | | (1H, d, ArH, J = 2.4), 8.32 (1H, d, Pyrimidine H, J = 5.4), | | | |
| | | 9.33 (1H, s, NH), 10.72 (1H, bs, Pyrrole NH) | | | |
| 45 | d6- | 2.20 (3H, s, CH₃), 2.43 (3H, s, CH₃), 3.09 (4H, m, | m/z (ES) | 13.02 | — |
| | DMSO | CH₂NCH₂), 3.68 (4H, m, CH₂OCH₂), 6.50 (1H, s, | 417.83 | | |
| | | Pyrrole NH), 6.86 (1H, d, Pyrimidine H, J = 5.4), 7.96 | (M − H) | | |
| | | (2H, s, ArH), 8.37 (1H, d, Pyrimidine H, J = 5.4), 9.59 | C₂₀H₂₁Cl₂N₅O = | | |
| | | (1H, s, NH), 10.77 (1H, s, Pyrrole NH) | 418.32 | | |
| 46 | d6- | 1.29 (3H, t, CH₃, J = 7.2), 2.35 (3H, s, CH₃), 2.41 (3H, s, | m/z (ES) | 18.95 | — |
| | DMSO | CH₃), 2.90 (4H, m, CH₂NCH₂), 3.73 (4H, m, | 455.88 | | |
| | | CH₂OCH₂), 4.25 (2H, q, OCH₂), J = 7.2), 6.82 (1H, d, | C₂₃H₂₆ClN₅O₃ = | | |
| | | Pyrimidine H, J = 5.4), 7.11 (1H, d, ArH, J = 8.8), 7.58 | 455.94 | | |
| | | (1H, dd, ArH, J = 8.8, 2.4), 8.08 (1H, d, ArH, J = 2.4), | | | |
| | | 8.41 (1H, d, Pyrimidine H, J = 5.4), 9.50 (1H, s, NH), | | | |
| | | 11.69 (1H, bs, Pyrrole NH) | | | |
| 47 | d6- | 1.29 (3H, t, CH₃, J = 7.3), 2.42 (3H, s, CH₃), 2.45 (3H, s, | m/z (ES) | 24.58 | — |
| | DMSO | CH₃), 3.07 (4H, m, CH₂NCH₂), 3.68 (4H, m, | 489.89 | | |
| | | CH₂OCH₂), 4.25 (2H, q, OCH₂), J = 7.2), 6.88 (1H, s, | (M − H) | | |
| | | Pyrrole H), 6.90 (1H, d, Pyrimidine H, J = 5.4), 7.95 (1H, | C₂₃H₂₅Cl₂N₅O₃ = | | |
| | | s, ArH), 8.47 (1H, d, Pyrimidine H, J = 5.4), 9.76 (1H, s, | 490.38 | | |
| | | NH), 11.73 (1H, bs, Pyrrole NH) | | | |
| 48 | d6- | 2.12 (3H, s, CH₃), 2.38 (3H, s, CH₃), 3.01 (4H, m, | m/z (ES) | 15.79 | — |
| | DMSO | CH₂NCH₂), 3.47 (3H, s, NCH₃), 3.73 (4H, m, | 364.05 | | |
| | | CH₂OCH₂), 6.51 (1H, s, Pyrrole H), 6.67 (1H, d, | (M + H) | | |
| | | Pyrimidine H, J = 5.4), 6.86 (2H, d, ArH, J = 8.8), 7.62 | C₂₁H₂₅N₅O = | | |
| | | (2H, d, ArH, J = 8.8), 8.27 (1H, d, Pyrimidine H, J = 5.4), | 363.46 | | |
| | | 9.03 (1H, s, NH) | | | |
| 49 | d6- | 2.14 (3H, s, Ch₃), 2.41 (3H, s, CH₃), 2.90 (4H, m, | m/z (ES) | 19.71 | — |
| | DMSO | CH₂NCH₂), 3.49 (3H, s, NCH₃), 3.72 (4H, m, | 398.07 | | |
| | | CH₂OCH₂), 6.53 (1H, s, Pyrrole H), 6.75 (1H, d, | (M + H) | | |
| | | Pyrimidine H, J = 5.4), 7.10 (1H, d, ArH, J = 8.8), 7.59 | C₂₁H₂₄ClN₅O = | | |
| | | (1H, dd, ArH, J = 2.0), 8.10 (1H, d, ArH, J = 2.0), 8.33 | 397.90 | | |
| | | (1H, d, Pyrimidine H, J = 5.4), 9.38 (1H, s, NH) | | | |
| 50 | d6- | 2.07 (3H, s, CH₃), 2.16 (3H, s, CH₃), 2.24 (3H, s, CH₃), | m/z (ES) | 11.20* | — |
| | DMSO | 3.03-3.06 (4H, m, 2 × CH₂), 3.72-3.75 (4H, m, 2x | 363.99 | | |
| | | CH₂), 6.38 (1H, s, CHNH), 6.62 (1H, d, Pyrimidine H, | C₂₁H₂₅N₅O = | | |
| | | J = 5.5), 6.73 (1H, dd, ArH, J = 9.0, 3.0), 6.79 (1H, d, | 363.46 | | |
| | | ArH, J = 3.0), 7.24 (1H, d, ArH, J = 9.0), 8.15 (1H, d, | | | |
| | | Pyrimidine H, J = 5.5), 8.16 (1H, s, CHNH) 10.58 (1H, s, | | | |
| | | NH) | | | |
| 51 | d6- | 1.21 (6H, d, 2 × OCHCH₃, J = 6.5), 2.17 (3H, s, CH₃), 2.50 | m/z (ES) | 12.93* | — |
| | DMSO | (3H, s, CH₃), 2.74 (2H, dd, 2 × CHH, J = 11.5, 6.0), 3.07 | 377.99 | | |
| | | (2H, dd, 2 × CHH, J = 11.5, 3.0), 4.02-4.06 (2H, m, | C₂₂H₂₇N₅O = | | |
| | | 2 × CHCH₃), 6.45 (1H, s, Pyrrole H), 6.68 (1H, d, | 377.48 | | |
| | | Pyrimidine H, J = 5.5), 6.84 (2H, d, 2 × ArH, J = 9.0), 7.60 | | | |
| | | (2H, d, 2 × ArH, J = 9.0), 8.25 (1H, d, Pyrimidine H, | | | |
| | | J = 5.5), 8.96 (1H, s, NH), 10.66 (1H, s, Pyrrole NH) | | | |
| 52 | d6- | 2.32 (3H, s, CH₃), 2.42 (3H, s, CH₃), 3.61 (3H, s, | m/z (ES) | 16.32 | — |
| | DMSO | OCH₃), 3.73 (6H, s, 2 × OCH₃), 6.82 (1H, d, Pyrimidine | 377.99 | | |
| | | H, J = 4.9), 7.16 (2H, s, ArH), 8.42 (1H, d, Pryimidine H, | (M − H) | | |
| | | J = 4.9), 9.26 (1H, s, NH), 12.19 (1H, bs, Pyrrole NH) | C₂₀H₂₁N₅O = | | |
| | | | 379.41 | | |
| 53 | d6- | 2.30 (3H, s, CH₃), 2.44 (3H, s, CH₃), 3.61 (3H, s, | m/z (ES) | 17.71 | — |
| | DMSO | NCH₃), 3.64 3H, s, OCH₃), 3.73 (6H, s, 2xOCH₃), 6.81 | 393.97 | | |
| | | (1H, d, Pyrimidine CH, J = 5.4), 7.17 (2H, s, ArH), 8.44 | C₂₁H₂₃N₅O₃ = | | |
| | | (1H, d, Pyrimidine CH, J = 5.4), 9.32 (1H, s, NH) | 393.92 | | |
| 54 | d6- | 1.31 (3H, t, CH₃, J = 7.3), 2.39 (3H, s, CH₃), 2.43 (3H, s, | m/z (ES) | 15.33 | — |
| | DMSO | CH₃), 2.89 (4H, m, CH₂NCH₂), 3.70 (4H, m, | 451.98 | | |
| | | CH₂OCH₂), 3.74 (3H, s, OCH₃), 4.24 (2H, q, CO₂CH₂, | C₂₄H₂₉N₅O₄ = | | |
| | | J = 7.3), 6.75 (1H, d, Pyrimidine H, J = 4.9), 6.82 (1H, d, | 451.52 | | |
| | | ArH, J = 8.8), 7.36 (1H, s, ArH), 7.38 (1H, d, ArH, | | | |
| | | J = 8.8), 8.37 (1H, d, Pyrimidine H, J = 4.9), 9.21 (1H, s, | | | |
| | | NH), 11.64 (1H, bs, Pyrrole NH) | | | |
| 55 | d6- | 1.28 (3H, t, CH₃, J = 7.3), 2.40 (3H, s, CH₃), 2.44 (3H, s, | m/z (ES) | 19.80 | — |
| | DMSO | CH₃), 3.70 (6H, s, 2 × OCH₃), 4.24 (2H, q, CO₂CH₂, | 396.98 | | |
| | | J = 7.3), 6.10 (1H, d, ArH, J = 2.4), 6.82 (1H, d, | C₂₁H₂₄N₄O₄ = | | |
| | | Pyrimidine H, J = 5.4), 7.10 (1H, d, ArH, J = 2.4), 8.42 | 396.44 | | |
| | | (1H, d, Pyrimidine H, J = 5.4), 9.36 (1H, s, NH), 11.67 | | | |
| | | (1H, bs, Pyrrole NH) | | | |
| 56 | d6- | 2.19 (3H, s, CH₃), 2.41(3H, s, CH₃), 3.61 (3H, s, | m/z (ES) | 16.22 | — |
| | DMSO | OCH₃), 3.73 (6H, s, 2 × OCH₃), 6.46 (1H, d, Pyrrole H, | 354.97 | | |
| | | J = 1.0), 6.75 (1H, d, Pyrimidine H, J = 5.4), 7.19 (2H, s, | C₁₉H₂₂N₄O₃ = | | |
| | | ArH), 8.31 (1H, d, Pyrimidine H, J = 5.4), 9.05 (1H, s, | 354.40 | | |
| | | NH), 10.68 (1H, bs, Pyrrole NH) | | | |
| 57 | d6- | 2.14 (3H, s, CH₃), 2.40 (3H, s, CH₃), 3.44 (3H, s, | m/z (ES) | 17.73 | — |
| | DMSO | NCH₃), 3.59 (3H, s, OCH₃), 3.73 (6H, s, 2 × OCH₃), 6.54 | 369.04 | | |
| | | (1H, s, Pyrrole H), 6.72 (1H, d, Pyrimidine H, J = 5.4), | (M + H) | | |
| | | 7.2 (2H, s, ArH), 8.32 (1H, d, Pyrimidine H, J = 5.4), | C₂₀H₂₄N₄O₃ = | | |
| | | 9.11 (1H, s, NH) | 368.43 | | |
| 58 | d6- | 1.30 (3H, t, CH₃, J = 7.3), 2.40 (3H, s, CH₃), 2.44 (3H, | m/z (ES) | 17.93 | — |
| | DMSO | s, CH₃), 3.61 (3H, s, OCH₃), 3.73 (6H, s, 2xOCH₃), 4.25 | 426.91 | | |
| | | (2H, q, CO₂CH₂, J = 7.3), 6.79 (1H, d, Pyrimidine H, | C₂₂H₂₆N₄O₅ = | | |
| | | J = 4.9), 7.19 (2H, s, ArH), 8.39 (1H, d, Pyrimidine H, | 426.47 | | |
| | | J = 4.9), 9.24 (1H, s, NH), 11.65 (1H, bs, Pyrrole NH) | | | |
| 59 | d6- | 2.18 (3H, s, CH₃), 2.40 (3H, s, CH₃), 2.89 (4H, m, | m/z (ES) | 9.92* | — |
| | DMSO | CH₂NCH₂), 3.70 (4H, m, CH₂OCH₂), 3.74 (3H, s, | 380.04 | | |
| | | OCH₃), 6.45 (1H, s, Pyrrole H), 6.72 (1H, d, Pyrimidine | (M + H) | | |
| | | H, J = 5.5), 6.79 (1H, d, Ar H, J = 8.0), 7.39 (1H, d, ArH, | C₂₁H₂₅N₅O₂ = | | |
| | | J = 8.0), 7.40 (1H, s, ArH), 8.28 (1H, d, Pyrimidine H, | 379.46 | | |
| | | J = 5.5), 9.01 (1H, s, NH) 10.68 (1H, s, Pyrrole NH) | | | |
| 60 | d6- | 2.03 (3H, s, CH₃), 2.17 (3H, s, CH₃), 2.39 (3H, s, CH₃), | m/z (ES) | 10.76* | — |
| | DMSO | 2.97-3.00 (2H, m, 2 × CHH), 3.04-3.07 (2H, m, 2 × CHH), | 390.84 | | |
| | | 3.54-3.58 (4H, m, 2 × CH₂), 6.45 (1H, s, Pyrrole H), 6.69 | C₂₂H₂₆N₆O = | | |
| | | (1H, d, Pyrimidine H, J = 5.0), 6.89 (2H, d, 2 × ArH, | 390.48 | | |
| | | J = 9.0), 7.62 (2H, d, 2 × ArH, J = 9.0), 8.25 (1H, d, | | | |
| | | Pyrimidine H, J = 5.0), 8.99 (1H, s, NH) and 10.67 (1H, | | | |
| | | s, Pyrrole NH). | | | |

*= 10-70-20 HPLC gradient
^{#}= 0-60-20 HPLC gradient

TABLE III
In Vitro Kinase Data for Selected Compounds of the Invention

| | In vitro kinase assays | | |
|---|---|---|---|
| | Ki (microM) | | |
| | CDK2/ | | Dm |
| No. | E | GSK3 | AK-A |
| 1 | | 0.601 | >10 |
| 2 | | 3.69 | >10 |
| 4 | 1.81 | | 0.008 |
| 6 | | | 0.472 |
| 7 | | | 0.423 |
| 8 | | | >10 |
| 9 | | | >10 |
| 10 | | | >10 |
| 11 | | | 0.278 |
| 12 | | | 0.303 |
| 13 | | 0.208 | 0.335 |
| 15 | | 0.133 | 0.602 |
| 16 | | 0.045 | |
| 17 | 0.033 | 3.269 | 0.04 |
| 18 | 0.119 | 0.005 | |
| 19 | 0.041 | 0.004 | |
| 20 | 0.538 | 0.011 | |
| 24 | 0.574 | 0.127 | |
| 26 | 0.861 | 0.167 | |
| 27 | >10 | 0.375 | >10 |
| 28 | 0.188 | 0.005 | 0.177 |
| 29 | >10 | 0.731 | 2.523 |
| 30 | 0.197 | 0.003 | 0.681 |
| 31 | 0.119 | 0.053 | 0.049 |
| 32 | 0.377 | 0.003 | 0.161 |
| 33 | | 0.007 | 0.666 |
| 34 | 0.876 | 0.005 | 1.297 |
| 36 | 0.012 | 0.014 | 0.307 |
| 37 | | | 0.333 |
| 38 | 0.31 | | 0.069 |
| 39 | | | 0.091 |
| 40 | | | 0.108 |
| 41 | 0.308 | | 0.019 |
| 42 | | | 0.73 |
| 43 | | | 0.103 |
| 52 | 0.759 | | |
| 53 | 1.119 | | |
| 54 | >10 | | |
| 56 | >10 | | |
| 57 | >10 | | |
| 58 | 4.703 | | |

TABLE IV
Glycogen synthase activation in HEK293 cell, mouse
adipocytes (3T3-L1) and rat myotubes (L6.G8.C5)

| | Glycogen synthase activation EC50 (uM) | | |
|---|---|---|---|
| Compound | HEK293 | 3T3-L1 | L6.G8.C5 |
| 18 | 1.5 | 4.12 | ND |
| 19 | 0.7 | 5.6 | 6.3 |
| 28 | 6.6 | 7.9 | 9 |
| 30 | 2.2 | 4.3 | 2.3 |
| 32 | 4.1 | 2.1 | 5 |

TABLE V
PEPCK gene expression in HEPG2 cells - QPCR assay

| Compound | Concentration (uM) | Dex/cAMP | % PEPCK activity |
|---|---|---|---|
| — | — | − | 13.2 ± 1.7 |
| — | — | + | 100 |
| 19 | 1 | + | 72.9 ± 9.06 |
| 19 | 10 | + | 45.4 ± 1.9 |
| 28 | 1 | + | 48.4 ± 7.3 |
| 28 | 10 | + | 11.7 ± 1.3 |
| 30 | 1 | + | 22.8 ± 12.1 |
| 30 | 10 | + | 16.2 ± 3.8 |
| Insulin | 0.1 | + | 67.5 ± 9.0 |

1. A compound of formula I or II which is selected from the following:

| | |
|---|---|
| 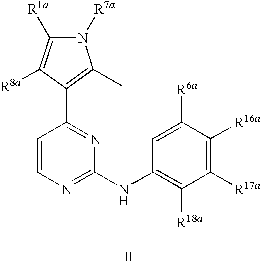 | 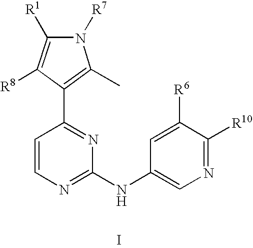 |
| Cpd | | | | | | | | |
|---|---|---|---|---|---|---|---|---|
| Structure | | R¹/R^{1a} | R⁷/R^{7a} | R⁸/R^{8a} | R⁶/R^{6a} | R¹⁰/R^{16a} | R^{17a} | R^{18a} |
| 1 | II | H | H | Me | H | CN | H | H |
| 2 | II | H | H | Me | H | H | CN | H |
| 3 | II | H | H | Me | H | H | CH₂OH | H |
| 4 | II | H | H | Me | H | Morph | H | H |
| 5 | II | CO₂Et | H | Ph | H | H | NO₂ | H |
| 6 | II | CO₂Et | H | Ph | H | OH | H | H |
| 7 | II | CO₂Et | H | Ph | H | H | OH | H |
| 8 | II | CO₂Et | H | Ph | H | H | F | H |
| 9 | II | CO₂Et | H | Ph | H | I | H | H |
| 10 | II | CO₂Et | H | Ph | H | CF₃ | H | H |
| 11 | II | CO₂Et | H | Ph | H | OH | NO₂ | H |
| 12 | II | CO₂Et | H | Ph | H | NMe₂ | H | H |
| 13 | II | H | H | Ph | H | H | NO₂ | H |
| 14 | II | H | H | Ph | H | I | H | H |
| 15 | II | H | H | Ph | H | OH | H | H |
| 16 | II | H | H | Ph | H | F | H | H |
| 17 | II | CH₂NMe₂ | H | Me | H | H | NO₂ | H |
| 18 | II | CN | H | Me | H | NMe₂ | NO₂ | H |
| 19 | II | NO₂ | H | Me | H | NMe₂ | NO₂ | H |
| 20 | II | CN | Me | Me | H | NMe₂ | H | H |
| 22 | II | CO₂Et | H | Ph | H | F | H | H |
| 24 | II | CN | H | Me | H | Morph | H | H |
| 26 | I | H | H | Me | H | OMe | — | — |
| 27 | II | CN | H | Me | NO₂ | H | H | Me |
| 28 | I | CN | H | Me | H | OMe | — | — |
| 29 | II | CN | H | Me | OMe | H | H | Me |
| 30 | I | CN | H | Me | H | Cl | — | — |
| 31 | II | CN | H | Me | H | NHMe | CF₃ | H |
| 32 | II | CN | H | Me | H | H | 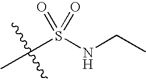 | H |
| 33 | I | CN | H | Me | Me | Cl | — | — |
| 34 | II | CN | H | Me | H | H | 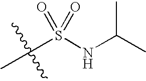 | H |
| 36 | II | CN | H | Me | H | 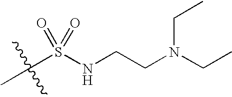 | H | H |
| 37 | II | CO₂(CH₂)₂OMe | H | Ph | H | Morph | H | H |
| 38 | I | CN | H | Me | H | N—Ac | — | — |
| | | | | | | Piperizine | | |
| 39 | I | CN | Me | Me | H | N—Ac | — | — |
| | | | | | | Piperizine | | |
| 40 | II | CN | H | Me | H | N-Bz | H | H |
| | | | | | | Piperizine | | |
| 41 | II | CN | H | Me | H | N—Ac | H | H |
| | | | | | | Piperizine | | |
| 42 | II | H | H | Ph | H | Morph | H | H |
| 43 | II | CO₂Et | H | Me | H | Morph | H | H |
| 44 | II | H | H | Me | H | Morph | Cl | H |
| 45 | II | H | H | Me | Cl | Morph | Cl | H |
| 46 | II | CO₂Et | H | Me | H | Morph | Cl | H |
| 47 | II | CO₂Et | H | Me | Cl | Morph | Cl | H |
| 48 | II | H | Me | Me | H | Morph | H | H |
| 49 | II | H | Me | Me | H | Morph | Cl | H |
| 50 | II | H | H | Me | H | Morph | H | Me |
| 51 | II | H | H | Me | H | bis-methyl | H | H |
| | | | | | | Morph | | |
| 52 | II | CN | H | Me | OMe | OMe | OMe | H |
| 53 | II | CN | Me | Me | OMe | OMe | OMe | H |
| 54 | II | CO₂Et | H | Me | H | Morph | OMe | H |
| 55 | II | CO₂Et | H | Me | OMe | H | OMe | H |
| 56 | II | H | H | Me | OMe | OMe | OMe | H |
| 57 | II | H | Me | Me | OMe | OMe | OMe | H |
| 58 | II | CO₂Et | H | Me | OMe | OMe | OMe | H |
| 59 | I. | H | H | Me | H | Morph | OMe | H |
| 60 | II | H | H | Me | H | N—Ac- | H | H |
| | | | | | | piperazine | | |

and pharmaceutically acceptable salts thereof.

2. A compound of formula I, or a pharmaceutically acceptable salt thereof, 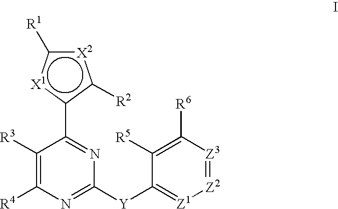
wherein
one of X¹ and X² is NR and the other is CR⁸;
one of Z¹, Z² and Z³ is N or NR⁹⁺ and the remainder are each independently CR¹⁰;
Y is selected from NR¹¹, NHCO, NHSO₂, NHCH₂, CH₂, CH₂CH₂, or CH═CH;
R¹-R⁶, R⁸ and each R¹⁰ are each independently selected from H or (CH₂)ₘR², where m is 0, 1, 2, or 3;
R⁷ and R¹¹ are each independently H or alkyl;
R⁹ is alkyl;
each R¹² is independently selected from OR¹³, R¹³, COR¹³, COOR¹³, CN, CONR¹³R¹⁴, NR¹³R¹⁴, NR¹³COR¹⁴, SR¹³, SOR¹³, SO₂R¹³, NR¹³SO₂R¹⁴, SO₂OR¹³, SO₂NR¹³R¹⁴, halogen, CF₃, and NO₂;
R¹³ and R¹⁴ are each independently H or (CH₂)ₙR¹⁵, where n is 0, 1, 2, or 3; and
each R¹⁵ is independently selected from alkyl, cycloalkyl, heteroaryl, aralkyl, aryl and heterocycloalkyl, each of which may be optionally substituted by one or more substituents selected from halogen, OH, CN, COO-alkyl, aralkyl, SO₂-alkyl, SO₂-aryl, COOH, CO-alkyl, CO-aryl, NH₂, NH-alkyl, N(alkyl)₂, CF₃, alkyl and alkoxy, wherein said alkyl and alkoxy groups may be further substituted by one or more OH groups.

3. A compound according claim 2 wherein Y is NR¹¹.

4. A compound according to claim 2 wherein Y is NH.

5. A compound according to claim 2 wherein R³ and R⁴ are both H.

6. A compound according to claim 2 wherein each R¹⁵ is independently selected from ethyl, ethyl, isopropyl, n-butyl, isobutyl, t-butyl, cyclopropyl, cyclobutyl, cyclopentyl, cyclohexyl, phenyl, pyridinyl, pyrrolidinyl, pyrrolyl, morpholinyl, piperazinyl, piperidinyl, triazolyl, tetrazolyl and thiazolyl.

7. A compound according to claim 2 wherein each R¹² is independently selected from OH, OMe, COMe, CHO, CO₂Me, COOH, CN, CONH₂, NHMe, NH₂, NMe₂, SH, SMe, SOMe, SO₂Me, SO₂NHMe, SO₂NH₂, Cl, Br, F, I, CF₃, NO₂, N-morpholinyl, N-pyrrolidinyl and N-piperazinyl.

8. A compound according to claim 2 wherein:
R⁵, R⁶ and each R¹⁰ are each independently selected from H and (CH₂)ₘR¹²;
each R¹² is independently selected from R¹³, NR¹³COR¹⁴, NR¹³R¹⁴, SO₂R¹³, NR¹³SO₂R¹⁴, OR¹³, alkyl, NO₂, CF₃, alkoxy, halogen;
R¹³ and R¹⁴ are each independently H or (CH₂)ₙR¹⁵; and
each R¹⁵ is independently selected from alkyl, heteroaryl, aryl and heterocycloalkyl, each of which may be optionally substituted by one or more substituents selected from halogen, OH, CN, COO-alkyl, COOH, CO-alkyl, aralkyl, SO₂-alkyl, SO₂-aryl, CO-aryl, alkyl, alkoxy, NH₂, NH-alkyl, N(alkyl)₂ and CF₃.

9. A compound according to claim 2 wherein:
R⁵, R⁶ and each R¹⁰ are each independently selected from H and R¹²;
each R¹² is independently selected from R¹³, NHCOR¹⁴, NR¹³R¹⁴, SO₂R¹³, NHSO₂R¹⁴, OR¹³, alkyl, NO₂, CF₃, alkoxy, halogen;
R¹³ and R¹⁴ are each independently H or R¹⁵; and
each R¹⁵ is independently selected from alkyl, aryl and heterocycloalkyl, each of which may be optionally substituted by one or more substituents selected from halogen, OH, CO-alkyl, aralkyl, SO₂-alkyl, SO₂-aryl, CO-aryl, alkyl, alkoxy, NH₂, NH-alkyl and N(alkyl)₂.

10. A compound according to claim 2 wherein R⁵, R⁶ and each R¹⁰ are each independently selected from H, Me, NO₂, CF₃, OMe, F, N-morpholinyl, N-piperazinyl and N-piperidinyl, said N-morpholinyl, N-piperazinyl and N-piperidinyl groups being optionally substituted by one or more substituents selected from halogen, OH, CN, COO-Me, COOH, CO-Me, CO-phenyl, Me, OMe, NH₂, NH-Me, NMe₂ and CF₃.

11. A compound according to claim 2 wherein X¹ is CR⁸ and X² is NR⁷.

12. A compound according to claim 11 wherein R⁷ is H or Me.

13. A compound according to claim 2 wherein R¹, R² and each R¹⁰ are each independently selected from H, CN, NO₂, alkyl, CONR¹³R¹⁴, NR¹³R¹⁴, NHCOR¹³OR¹³, R¹³, and NR¹³SO₂R¹⁴.

14. A compound according to claim 2 wherein R¹, R² and each R¹⁰ are each independently selected from H, CN, NO₂, alkyl, NR¹³R¹⁴, NR¹³COR¹⁴ and OR¹³, where R¹³ and R¹⁴ are each independently H or alkyl.

15. A compound according to claim 2 wherein:
X¹ is CR⁸;
X² is NR⁷;
R² and R⁸ are both alkyl;
R¹ is selected from H, CN, NO₂, alkyl, CONR¹³R¹⁴, NR¹³R¹⁴, NHCOR¹³OR¹³, R¹³, and NR³SO₂R¹⁴.

16. A compound according to claim 15 wherein R¹ is H or CN.

17. A compound according to claim 2 wherein Z² is N and Z¹ and Z³ are each independently CR¹⁰.

18. A compound according to claim 2 wherein R⁵, R⁶ and each R¹⁰ are each independently selected from H, halo, alkyl, alkoxy and heterocycloalkyl optionally substituted by one or more alkyl or acyl substituents.

19. A compound according to claim 2 wherein R⁵, R⁶ and each R¹⁰ are each independently selected from H, halo, alkyl, alkoxy, N-piperazinyl, N-morpholinyl and N-piperidinyl, wherein said N-piperazinyl, N-morpholinyl and N-piperidinyl groups are optionally substituted by one or more alkyl or acyl substituents.

20. A compound according to claim 2 wherein:
R⁵ is H;
R⁶ is H or alkyl;
Z¹ is CH;
Z² is N;
Z³ is CR¹⁰ where R¹⁰ is independently H, alkoxy, halo or N-piperazinyl, and where said N-piperazinyl group is optionally substituted by an acyl group.

21. A compound according to claim 2 which is selected from the following:

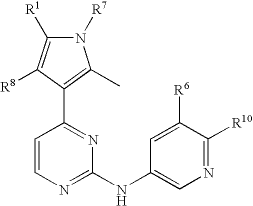

| Compound | R¹ | R⁶ | R⁷ | R⁸ | R¹⁰ |
|---|---|---|---|---|---|
| 26 | H | H | H | Me | OMe |
| 28 | CN | H | H | Me | OMe |
| 30 | CN | H | H | Me | Cl |
| 33 | CN | Me | H | Me | Cl |
| 38 | CN | H | H | Me | N—Ac Piperazine |
| 39 | CN | H | Me | Me | N—Ac Piperazine |

and pharmaceutically acceptable salts thereof.

22. A compound of formula II, or a pharmaceutically acceptable salt thereof, 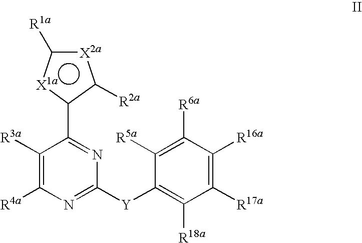
wherein
one of X^{1a} and X^{2a} is NR^{7a} and the other is CR^{8a};
Y is selected from NR^{11a}, NHCO, NHSO₂, NHCH₂, CH₂, CH₂CH₂, or CH═CH;
R^{1a}-R^{6a}, R^{8a}, R^{16a}, R^{17a} and R^{18a} are each independently selected from H or (CH₂)ₘR^{12a}, where m is 0, 1, 2, or 3;
R^{7a} and R^{11a} are each independently H or alkyl;
each R^{12a} is independently selected from OR^{13a}, R^{13a}, COR^{13a}, COOR^{13a}, CN, CONR^{13a}R^{14a}, NR^{13a}R^{14a}, NR^{13a}COR^{14a}SR^{13a}, SOR^{13a}, SO₂R^{13a}, NR^{13a}SO₂R^{14a}, SO₂OR^{13a}, SO₂NR^{13a}R^{14a}, halogen, CF₃, and NO₂;
R^{13a} and R^{14a} are each independently H or (CH₂)ₙR^{15a}, where n is 0, 1, 2, or 3; and
each R^{15a} is independently selected from alkyl, cycloalkyl, heteroaryl, aralkyl, aryl and heterocycloalkyl, each of which may be optionally substituted by one or more substituents selected from halogen, OH, CN, COO-alkyl, aralkyl, SO₂-alkyl, SO₂-aryl, COOH, CO-alkyl, CO-aryl, NH₂, NH-alkyl, N(alkyl)₂, CF₃, alkyl and alkoxy, wherein said alkyl and alkoxy groups may be further substituted by one or more OH groups;
wherein at least one of R^{1a}-R^{6a}, R^{8a}, R^{16a}, R^{17a} and R^{18a} is selected from SO₂NR^{13a}R^{14a} and optionally substituted heterocycloalkyl.

23. A compound according claim 22 wherein Y is NR^{11a}.

24. A compound according to claim 22 wherein Y is NH.

25. A compound according to claim 22 wherein R^{3a} and R^{4a} are both H.

26. A compound according to claim 22 wherein each R^{15a} is independently selected from ethyl, ethyl, isopropyl, n-butyl, isobutyl, t-butyl, cyclopropyl, cyclobutyl, cyclopentyl, cyclohexyl, phenyl, pyridinyl, pyrrolidinyl, pyrrolyl, morpholinyl, piperazinyl, piperidinyl, triazolyl, tetrazolyl and thiazolyl.

27. A compound according to claim 22 wherein each R^{12a} is independently selected from OH, OMe, COMe, CHO, CO₂Me, COOH, CN, CONH₂, NHMe, NH₂, NMe₂, SH, SMe, SOMe, SO₂Me, SO₂NHMe, SO₂NH₂, Cl, Br, F, I, CF₃, NO₂, N-morpholinyl, N-pyrrolidinyl and N-piperazinyl.

28. A compound according to claim 22 wherein:
R^{5a}, R^{6a}, R^{16a}, R^{17a} and R^{18a} are each independently selected from H and R^{12a},
each R^{12a} is independently selected from R^{13a}, NHCOR^{14a}, NR^{13a}R^{14a}, SO₂R^{13a}, NHSO₂R^{14a}, OR^{13a}, alkyl, NO₂, CF₃, alkoxy, halogen and SO₂NR^{13a}R^{14a};
R^{13a} and R^{14a} are each independently H or R^{15a}; and
each R^{15a} is independently selected from alkyl, aryl and heterocycloalkyl, each of which may be optionally substituted by one or more substituents selected from halogen, OH, CO-alkyl, aralkyl, SO₂-alkyl, SO₂-aryl, CO-aryl, alkyl, alkoxy, NH₂, NH-alkyl and N(alkyl)₂.

29. A compound according to claim 22 wherein R^{5a}, R^{6a}, R^{16a}, R^{17a} and R^{18a} are each independently selected from H, Me, NO₂, CF₃, OMe, F, SO₂NH-alkyl, N-morpholinyl, N-piperazinyl and N-piperidinyl, said alkyl, N-morpholinyl, N-piperazinyl and N-piperidinyl groups being optionally substituted by one or more substituents selected from halogen, OH, CN, COO-Me, COOH, CO-Me, CO-phenyl, Me, OMe, NH₂, NH-Me, NMe₂ and CF₃.

30. A compound according to claim 22 wherein X^{1a} is CR^{8a} and X²a is NR^{7a}.

31. A compound according to claim 30 wherein R^{7a} is H or Me.

32. A compound according to claim 22 wherein R^{1a}, R^{2a} and R^{8a} are each independently selected from H, CN, NO₂, alkyl, CONR^{13a}R^{14a}, NR^{13a}R^{14a}, NHCOR^{13a}OR^{13a}, R^{13a}, and NR^{13a}SO₂R^{14a}.

33. A compound according to claim 22 wherein R^{1a}, R^{2a} and R^{8a} are each independently selected from H, CN, NO₂, alkyl, NR^{13a}R^{14a}, NR^{13a}COR^{14a} and OR^{13a}, where R^{13a} and R^{14a} are each independently H or alkyl.

34. A compound according to claim 22 wherein:
X^{1a} is CR^{8a};
X^{2a} is NR^{7a};
R^{2a} is alkyl;
R^{8a} is alkyl or aryl;
R^{1a} is selected from H, CN, NO₂, COOR^{13a} alkyl, CONR^{13a}R^{14a}, NR^{13a}R^{14a}, NHCOR^{13a}OR^{13a}, R^{13a}, and NR^{13a}SO₂R^{14a}.

35. A compound according to claim 34 wherein R^{1a} is H, CN, CO₂(CH₂)₂OMe or CO₂Et.

36. A compound according to claim 22 wherein R^{5a}, R^{6a}, R^{16a}, R^{17a} and R^{18a} are each independently selected from H, halo, SO₂NR^{13a}R^{14a}, alkoxy and heterocycloalkyl optionally substituted by one or more alkyl, aralkyl or acyl substituents.

37. A compound according to claim 22 wherein R^{5a}, R^{6a}, R^{16a}, R^{17a} and R^{18a} are each independently selected from H, halo, SO₂NR^{13a}R^{14a}, alkoxy, N-piperazinyl, N-morpholinyl and N-piperidinyl, wherein said N-piperazinyl, N-morpholinyl and N-piperidinyl groups are optionally substituted by one or more alkyl, aralkyl or acyl substituents, and wherein R^{13a} and R^{14a} are each independently alkyl groups optionally substituted by one or more OH or alkoxy groups.

38. A compound according to claim 22 wherein R^{5a}, R^{6a}, R^{16a}, R^{17a} and R^{18a} are each independently selected from H, halo, alkoxy, SO₂NHEt, SO₂NHⁱPr, SO₂NHC(Me)₂CH₂OH, SO₂NHCH₂CH₂OMe, N-morpholinyl, N-Ac-piperazinyl, N-Bz-piperazinyl and bis-methyl-morpholinyl.

39. A compound according to claim 22 wherein:
R^{5a} and R^{18a} are H;
R^{6a} is H or halo;
R^{16a} and R^{17a} are each independently selected from H, halo, alkoxy, SO₂NHEt, SO₂NHⁱPr, SO₂NHC(Me)₂CH₂OH, SO₂NHCH₂CH₂OMe, N-morpholinyl, N-Ac-piperazinyl, N-Bz-piperazinyl and bis-methyl-morpholinyl.

40. A compound according to claim 22 which is selected from the following:

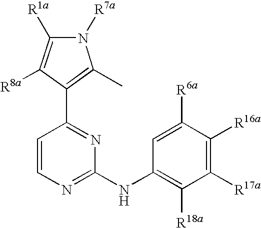

| Compound | R^{1a} | R^{7a} | R^{8a} | R^{6a} | R^{16a} | R^{17a} | R^{18a} |
|---|---|---|---|---|---|---|---|
| 4 | H | H | Me | H | Morph | H | H |
| 24 | CN | H | Me | H | Morph | H | H |
| 32 | CN | H | Me | H | H | 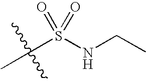 | H |
| 34 | CN | H | Me | H | H | 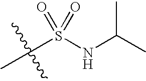 | H |
| 36 | CN | H | Me | H | 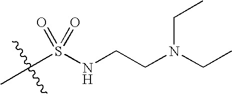 | H | H |
| 37 | CO₂(CH₂)₂OMe | H | Ph | H | Morph | H | H |
| 40 | CN | H | Me | H | N-Bz | H | H |
| | | | | | Piperizine | | |
| 41 | CN | H | Me | H | N—Ac | H | H |
| | | | | | Piperizine | | |
| 42 | H | H | Ph | H | Morph | H | H |
| 43 | CO₂Et | H | Me | H | Morph | H | H |
| 44 | H | H | Me | H | Morph | Cl | H |
| 45 | H | H | Me | Cl | Morph | Cl | H |
| 46 | CO₂Et | H | Me | H | Morph | Cl | H |
| 47 | CO₂Et | H | Me | Cl | Morph | Cl | H |
| 48 | H | Me | Me | H | Morph | H | H |
| 49 | H | | Me | H | Morph | Cl | H |
| 50 | H | H | Me | H | Morph | H | Me |
| 51 | H | H | Me | H | bis-methyl | H | H |
| | | | | | Morph | | |
| 54 | CO₂Et | H | Me | H | Morph | OMe | H |
| 59 | H | H | Me | H | Morph | OMe | H |
| 60 | H | H | Me | H | N—Ac— | H | H |
| | | | | | piperazine | | |

and pharmaceutically acceptable salts thereof.

41. A compound selected from the following:

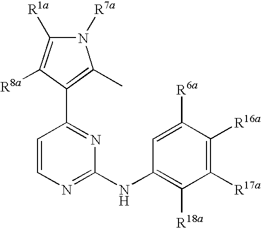

| Compound | R^{1a} | R^{7a} | R^{8a} | R^{6a} | R^{16a} | R^{17a} | R^{18a} |
|---|---|---|---|---|---|---|---|
| 1 | H | H | Me | H | CN | H | H |
| 2 | H | H | Me | H | H | CN | H |
| 3 | H | H | Me | H | H | CH₂OH | H |
| 5 | CO₂Et | H | Ph | H | H | NO₂ | H |
| 6 | CO₂Et | H | Ph | H | OH | H | H |
| 7 | CO₂Et | H | Ph | H | H | OH | H |
| 8 | CO₂Et | H | Ph | H | H | F | H |
| 9 | CO₂Et | H | Ph | H | I | H | H |
| 10 | CO₂Et | H | Ph | H | CF₃ | H | H |
| 11 | CO₂Et | H | Ph | H | OH | NO₂ | H |
| 12 | CO₂Et | H | Ph | H | NMe₂ | H | H |
| 13 | H | H | Ph | H | H | NO₂ | H |
| 14 | H | H | Ph | H | I | H | H |
| 15 | H | H | Ph | H | OH | H | H |
| 16 | H | H | Ph | H | F | H | H |
| 17 | CH₂NMe₂ | H | Me | H | H | NO₂ | H |
| 18 | CN | H | Me | H | NMe₂ | NO₂ | H |
| 19 | NO₂ | H | Me | H | NMe₂ | NO₂ | H |
| 20 | CN | Me | Me | H | NMe₂ | H | H |
| 22 | CO₂Et | H | Ph | H | F | H | H |
| 27 | CN | H | Me | NO₂ | H | H | Me |
| 29 | CN | H | Me | OMe | H | H | Me |
| 31 | CN | H | Me | H | NHMe | CF₃ | H |
| 52 | CN | H | Me | OMe | OMe | OMe | H |
| 53 | CN | Me | Me | OMe | OMe | OMe | H |
| 55 | CO₂Et | H | Me | OMe | H | OMe | H |
| 56 | H | H | Me | OMe | OMe | OMe | H |
| 57 | H | Me | Me | OMe | OMe | OMe | H |
| 58 | CO₂Et | H | Me | OMe | OMe | OMe | H |

and pharmaceutically acceptable salts thereof.

42. A pharmaceutical composition comprising a compound according to claim 2 admixed with a pharmaceutically acceptable diluent, excipient or carrier.

43. (canceled)

44. The method according to claim 58 wherein the proliferative disorder is cancer or leukaemia.

45. The method according to claim 58 wherein the proliferative disorder is glomerulonephritis, rheumatoid arthritis, psoriasis or chronic obstructive pulmonary disorder.

46. The method according to claim 58 wherein said compound is administered in combination with one or more other anticancer compounds.

47. (canceled)

48. The method according to claim 59 wherein the viral disorder is selected from human cytomegalovirus (HCMV), herpes simplex virus type 1 (HSV-1), human immunodeficiency virus type 1 (HIV-1), and varicella zoster virus (VZV).

49. A method for inhibiting a protein kinase comprising contacting a protein kinase with a compound according to any one of claims 1, 2, 22 and 41, such that said protein kinase is inhibited.

50. The method according to claim 49 wherein said protein kinase is a cyclin dependent kinase.

51. The method according to claim 50 wherein said cyclin dependent kinase is selected from CDK2, CDK7, CDK8 and CDK9.

52. The method according to claim 49 wherein said protein kinase is aurora kinase.

53. The method according to claim 52 wherein said aurora kinase is aurora kinase A, aurora kinase B or aurora kinase C.

54. The method according to claim 49 wherein said protein kinase is a tyrosine kinase.

55. The method according to claim 54 wherein said tyrosine kinase is Ableson tyrosine kinase (BCR-ABL), FMS-related tyrosine kinase 3 (FLT3), platelet-derived growth factor (PDGF) receptor tyrosine kinase or vascular endothelial growth factor (VEGF) receptor tyrosine kinase.

56. The method according to claim 49 wherein said protein kinase is GSK.

57. The method according to claim 56 wherein said protein kinase is GSK-3β.

58. A method of treating a proliferative disorder, said method comprising administering to a mammal a therapeutically effective amount of a compound according to claim 2, such that said proliferative disorder is treated.

59. A method of treating a viral disorder, said method comprising administering to a mammal a therapeutically effective amount of a compound according to claim 2, such that said viral disorder was treated.

60. A method of identifying further candidate compounds capable of inhibiting one or more of a cyclin dependent kinase, an aurora kinase, GSK, a tyrosine kinase, and a PLK enzyme, comprising using a compound according to claim 2 in an assay for identifying further candidate compounds capable of inhibiting one or more of a cyclin dependent kinase, an aurora kinase, GSK, a tyrosine kinase and a PLK enzyme.

61. The method according to claim 60 wherein said assay is a competitive binding assay.

62. The method according to claim 61 wherein said competitive binding assay comprises contacting said compound with an enzyme selected from a cyclin dependent kinase, GSK, a tyrosine kinase and PLK, and a candidate compound and detecting any change in the interaction between the compound and the enzyme.

63. A method for treating a CNS disorder, said method comprising administering to a mammal a therapeutically effective amount of a compound according to anyone of claims 1, 2, 22 and 41 such that said CNS disorder is treated.

64. The method according to claim 63 wherein the CNS disorder is Alzheimer's disease or bipolar disorder.

65. A method for treating alopecia, said method comprising administering to a mammal a therapeutically effective amount of a compound according to claim 2, such that said alopecia is treated.

66. A method for treating a stroke, said method comprising administering to a mammal a therapeutically effective amount of a compound according to claim 2, such that said stroke is treated.

67. The method according to claim 58 wherein the compound is administered in an amount sufficient to inhibit at least one PLK enzyme.

68. The method according to claim 67 wherein the PLK enzyme is PLK1.

69. The method according to claim 58 wherein the compound is administered in an amount sufficient to inhibit at least one CDK enzyme.

70. The method according to claim 69 wherein the CDK enzyme is CDK1, CDK2, CDK3, CDK4, CDK6, CDK7, CDK8 and/or CDK9.

71. The method according to claim 58 wherein the compound is administered in an amount sufficient to inhibit aurora kinase.

72. The method wherein the aurora kinase is aurora kinase A, aurora kinase B or aurora kinase C.

73. The method according to claim 58 wherein the compound is administered in an amount sufficient to inhibit at least one tyrosine kinase.

74. The method according to claim 73 wherein the tyrosine kinase is Ableson tyrosine kinase (BCR-ABL), FMS-related tyrosine kinase 3 (FLT3), platelet-derived growth factor (PDGF) receptor tyrosine kinase or vascular endothelial growth factor (VEGF) receptor tyrosine kinase.

75. A method for treating diabetes said method comprising administering to a mammal a therapeutically effective amount of a compound according to claim 2, such that said diabetes is treated.

76. The method according to claim 75 wherein the diabetes is Type II diabetes.

77. The method according to claim 75 wherein the compound is administered in an amount sufficient to inhibit GSK.

78. The method according to claim 76 wherein the compound is administered in an amount sufficient to inhibit GSK3β.

79. (canceled)

80. A method for treating an ophthalmic disorder, said method comprising administering to a mammal a therapeutically effective amount of a compound according to claim 2 such that said ophthalmic disorder is treated.

81. The method according to claim 80 wherein the ophthalmic disorder is glaucoma, exudative age-related macular degeneration (AMD) or proliferative diabetic retinopathy (PDR).

82. (canceled)
